(19) United States
(12) Patent Application Publication
(10) Pub. No.: US 2024/0279720 A1
(43) Pub. Date: Aug. 22, 2024

(54) USE OF GENETIC AND EPIGENETIC MARKERS TO DETECT CELL DEATH

(71) Applicant: Georgetown University, Washington, DC (US)

(72) Inventors: Anton Wellstein, Washington, DC (US); Megan E. McNamara, Washington, DC (US); Alexander H.K. Kroemer, Arlington, VA (US)

(73) Assignee: Georgetown University, Washington, DC (US)

(21) Appl. No.: 18/291,091

(22) PCT Filed: Jul. 25, 2022
(86) PCT No.: PCT/US2022/038242

§ 371 Date: Jan. 22, 2024

Related U.S. Application Data
(60) Provisional application No. 63/224,873, filed on Jul. 23, 2021.
(60) Provisional application No. 63/324,112, filed on Mar. 27, 2022.

(51) Int. Cl. C12Q 1/6827; A61K 45/06; C12Q 1/6881

(52) U.S. Cl. CPC C12Q 1/6827; A61K 45/06; C12Q 1/6881; C12Q 2600/154; C12Q 2600/156

(57) ABSTRACT

A method of detecting donor cell death in a subject receiving foreign biological material from a donor. The method comprises sequencing cfDNA in a biospecimen from the subject; determining cellular origin of the cfDNA by identifying methylation patterns in the sequence of the cfDNA and comparing the methylation patterns in the sequence of the cfDNA to known methylation patterns associated with different cell types; and determining source origin of the cfDNA by genotyping the cfDNA and identifying whether the cfDNA originates from the foreign biological material or from the subject. Cell death is detected when the cfDNA has both a cellular origin of the type of foreign biological material that was received from the donor, and a source origin of the donor.

### CROSS-REFERENCE TO RELATED APPLICATIONS

This application claims the benefit of U.S. Provisional Application No. 63/224,873, filed on Jul. 23, 2022, and U.S. Provisional Application No. 63/324,112, filed on Mar. 27, 2022, each of which is incorporated herein by reference in its entirety.

### STATEMENT REGARDING FEDERALLY SPONSORED RESEARCH OR DEVELOPMENT

This invention was made with government support under grant numbers T32 CA009686, F30 CA250307, and ROI CA231291 awarded by the National Institutes of Health. The government has certain rights in the invention.

### BACKGROUND OF THE INVENTION

DNA methylation is an important epigenetic regulation mechanism that comprises an addition of a methyl group to carbon 5 of cytosine residues in clusters of CpG islands. The effects of DNA methylation for each genomic locus is not fully understood, but it is widely accepted that DNA methylation can regulate gene expression (Yin et al., 2017), cellular differentiation and pathology (Shiels et al., 2017; Web and Guerau-de-Arellano. 2017; Si et al., 2015). DNA methylation is tissue-and cell type-specific (Bergman and Cedar, 2013; Bock, 2012; Fernandez et al., 2011) and can thus serve as a biomarker for specific tissues (Crowley et al., 2013). One application of DNA-methylation profiling has been used to predict the tissue of origin of cancers with unknown primary lesions. This has also been applied in clinical diagnostics and histopathology (Moran et al., 2017; Guo et al., 2017). DNA methylation signatures of tissues or cells can be obtained from ever growing methylome data bases (Plongthongkum et al., 2014) such as the Human Epigenome Atlas (www.genboree.org/epigenomeatlas/index.html) that is housed at Baylor College or from the published literature (Lehmann-Werman et al., 2016). In addition to isolation from cells and tissues, methylated DNA has been isolated from serum or plasma and was used to assess cancer progression, tissue transplant survival, prenatal diagnostics, and other phenotypes (Guo et al., 2017; Sun et al., 2015; Lo and Lam, 2016; Ciernia and LaSalle, 2016; Park et al., 2014; Dietrich, 2018; Yokoi et al., 2017). Short fragments of methylated DNA were reported in the circulation and in other biofluids: Saliva was used to identify altered neurotransmission in attention-deficit/hyperactivity disorder children via methylated DNA analysis (Wilmot et al., 2016).

Recent work has shown that after different modes of damage to normal tissues including inflammation (multiple sclerosis, pancreatitis), trauma (traumatic brain injury), or hypoxia (cardiac arrest) killing cells in the brain or peripheral nervous system, the pancreas etc. will release their DNA as short fragments into the circulation (Lehmann-Werman et al., 2016). This will lead to an increased abundance of tissue-specific DNA and the appropriate DNA methylation patterns can be used to identify the tissue or cellular origin of the circulating methylated DNA (cmeDNA) (id.)

Cell-free DNA (cfDNA) is released into the circulation as a result of cell turnover and can reflect the ongoing processes of systemic cell death and changes to homeostasis throughout the human body. Tissue and cell-type specific DNA methylation can be used for Tissue-of-Origin analysis to trace each cfDNA molecule make to its cellular origins and monitor altered tissue damage through the analysis of blood samples.

Identifying and measuring cell death and tissue damage is particularly significant in subjects receiving grafts or organs from donors. Donor cells may not survive due to a variety of mechanisms such as immune-mediated death, apoptosis, necrosis secondary to the trauma associated with transplantation, or bacterial or viral infection. As a result, methods of detecting and quantifying donor cell death can be invaluable for evaluating whether the subject is accepting the graft or organ.

### SUMMARY OF INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The invention provides a novel method for identifying and quantifying tissue-specific cell death from cfDNA.

In one aspect, the present invention relates to a method of detecting donor cell death in a subject receiving foreign biological material from a donor. The method comprises (a) sequencing cfDNA in a biospecimen from the subject; (b) determining cellular origin of the cfDNA by identifying methylation patterns in the sequence of the cfDNA and comparing the methylation patterns in the sequence of the cfDNA to known methylation patterns associated with different cell types; and (c) determining source origin of the cfDNA by genotyping the cfDNA and identifying whether the cfDNA originates from the foreign biological material or from the subject. Cell death is detected when the cfDNA has both a cellular origin of the type of foreign biological material that was received from the donor, and a source origin of the donor.

In another aspect, the present invention relates to a method of monitoring a subject's response to receiving foreign biological material from a donor, the method comprising detecting cell death in the subject at one or more time points after receiving the foreign biological material. Detection of cell death comprises: (a) sequencing cfDNA in a biospecimen from the subject; (b) determining cellular origin of the cfDNA by identifying methylation patterns in the sequence of the cfDNA and comparing the methylation patterns in the sequence of the cfDNA to known methylation patterns associated with different cell types; and (c) determining source origin of the cfDNA by genotyping the cfDNA and identifying whether the cfDNA originates from the foreign biological material or from the subject. Cell death is detected when the cfDNA has both a cellular origin of the type of foreign biological material that was received from the donor, and a source origin of the donor.

In yet another aspect, the present invention relates to a method of treating donor cell death in a subject receiving foreign biological material from a donor, the method comprising administering a treatment for donor cell death when donor cell death in the subject is detected, in which donor cell death is detected by a method comprising (a) sequencing cfDNA in a biospecimen from the subject; (b) determining cellular origin of the cfDNA by identifying methylation patterns in the sequence of the cfDNA and comparing the methylation patterns in the sequence of the cfDNA to known methylation patterns associated with different cell types; and (c) determining source origin of the cfDNA by genotyping the cfDNA and identifying whether the cfDNA originates from the foreign biological material or from the subject. Donor cell death is detected when the cfDNA has both a cellular origin of the type of foreign biological material that was received from the donor, and a source origin of the donor.

In a further aspect, the present invention relates to a method of treating donor cell death in a subject receiving foreign biological material from a donor, the method comprising administering a treatment for donor cell death when the quantity of donor cell death is increased between two or more time points after the subject receives the foreign biological material. Donor cell death is quantified by a method comprising (i) detecting donor cell death in the subject, in which detection of donor cell death comprises: (a) sequencing cfDNA in a biospecimen from the subject; (b) determining cellular origin of the cfDNA by identifying methylation patterns in the sequence of the cfDNA and comparing the methylation patterns in the sequence of the cfDNA to known methylation patterns associated with different cell types; and (c) determining source origin of the cfDNA by genotyping the cfDNA and identifying whether the cfDNA originates from the foreign biological material or from the subject; in which donor cell death is detected when the cfDNA has a cellular origin of the type of foreign biological material that was received from the donor, and has a source origin of the donor; and (ii) quantifying the cfDNA that has both a cellular origin of the type of foreign biological material that was received from the donor, and a source origin of the donor.

In some embodiments, the biospecimen comprises a biological fluid. In certain embodiments, the biological fluid is selected from blood, serum, plasma, cerebrospinal fluid, saliva, urine, and sputum. In preferred embodiments, the biological fluid comprises blood, serum, or plasma.

In some embodiments, the foreign biological material comprises liver tissue, cardiac tissue. vascular tissue, pancreatic tissue, splenic tissue, esophageal tissue, gastric tissue, intestinal tissue, colon tissue, lung tissue, tracheal tissue, skin tissue, subcutaneous tissue, hair tissue, kidney tissue, connective tissue, muscular tissue, skeletal tissue, cartilage tissue, prostate tissue, bladder tissue, gonadal tissue, uterine tissue, penile tissue, neural tissue, corneal tissue, ophthalmologic tissue, bone marrow tissue, and a population of blood-derived cells. In certain embodiments, the foreign biological material comprises liver tissue.

In some embodiments, the methylation pattern comprises a segment of nucleotide sequence containing at least 3 CpG dinucleotides.

In some embodiments, the cell types are selected from mature B-cell, naïve B-cell, biliary epithelial cell, breast basal cell, breast luminal cell, bulk endothelial cell, bulk epithelial cell, bulk immune cell, cardiomyocyte, cardiopulmonary endothelial cell, colon epithelial cell, dermal epithelial cell, granulocyte, hepatocyte, keratinocyte, kidney epithelial cell, liver endothelial cell, liver stromal cell, liver resident immune cell, lung epithelial cell, megakaryocyte, monocyte, macrophage, neuron, natural killer cell, pancreatic cell, prostate epithelial cell, skeletal muscular cell, and mature T-cell. In certain embodiments, the known methylation patterns are set forth in Table 2.

In some embodiments, genotyping the cfDNA comprises obtaining a polymorphic marker profile of the cfDNA and comparing it to a polymorphic marker profile obtained from the subject or the donor. The polymorphic marker profile may comprise polymorphic markers selected from single nucleotide polymorphisms, restriction fragment length polymorphisms, variable number of tandem repeats, short tandem repeats, hypervariable regions, minisatellites, dinucleotide repeats, trinucleotide repeats, tetranucleotide repeats, and simple sequence repeats. In certain embodiments, the polymorphic marker profile comprises polymorphic markers selected from single nucleotide polymorphisms

In some embodiments, determining cellular origin of the cfDNA comprises identifying methylation patterns in one or more portions of the sequence of the cfDNA. In certain embodiments, the polymorphic profile is obtained for the same one or more portions of the sequence of the cfDNA of which methylation patterns were identified. Cell death may be detected when the one or more portions of cfDNA has both a cellular origin of the type of foreign biological material that was received from the donor, and a source origin of the donor.

In some embodiments, the treatment comprises an immunosuppressive agent, an anti-inflammatory agent, an antibacterial therapy, an antiviral therapy, or a therapy targeted to a pathway that controls cell death.

In some embodiments, the cfDNA is quantified using chromatography, electrophoresis, comparative genomic hybridization, microarrays, or bead arrays.

In some embodiments, the increase in quantity of donor cell death between the two or more time points is at least 2-fold.

In some embodiments, the two or more time points are two or more days between Day 0 and Day 60 or at later time points with symptoms of tissue dysfunction after the subject receives the foreign biological material.

### BRIEF DESCRIPTION OF THE DRAWING FIGURES

FIG. 1 shows detection of donor-immune cell death in the circulation after transplant, as described in Example 1.

FIG. 2 shows levels of hepatocyte cfDNA in patients undergoing a liver transplant, as described in Example 2. The results are indicated for those patients who have for-cause (FC) graft dysfunction, and for those patients who do not have FC graft dysfunction. Hepatocyte cfDNA was measured on Day 0 before the transplantation (“Pre”). on Day 0 after the transplantation (“POST”), and on Days 7 (“POD7”) and 30 (“POD30”).

FIG. 3 shows dynamics of liver cell-type damages post-transplantation, as described in Example 2. The results show levels of hepatocyte, endothelial, stellate, and resident immune cfDNA of patients with liver damage and graft dysfunction (Panels A and B) and liver damage without graft dysfunction (Panels C and D), cfDNA was measured on Day 0 before the transplantation (“Pre”), on Day 0 after the transplantation (“POST”), and on Days 7 (“POD7”) and 30 (“POD30”).

FIG. 4 shows levels of endothelial cfDNA in patients undergoing a liver transplant, as described in Example 2. The results are indicated for those patients who have FC graft dysfunction, and for those patients who do not have FC graft dysfunction. Hepatocyte cfDNA was measured on Day 0 before the transplantation (“Pre”), on Day 0 after the transplantation (“POST”), and on Days 7 (“POD7”) and 30 (“POD30”).

### DETAILED DESCRIPTION OF THE INVENTION

The practice of the present invention can employ, unless otherwise indicated, conventional techniques of genetics, molecular biology, computational biology, genomics, epigenomics, mass spectrometry, and bioinformatics, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International d'Unités (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes ±10% of the recited value. For example, a concentration of about 1 mg/mL includes 0.9 mg/ml to 1.1 mg/mL. Likewise, a concentration range of about 1% to 10% (w/v) includes 0.9% (w/v) to 11% (w/v).

As used herein, the terms “cell-free DNA” or “cfDNA” or “circulating cell-free DNA” refers to DNA that is circulating in the peripheral blood of a subject. The DNA molecules in cfDNA may have a median size that is no greater than 1 kb (for example, about 50 bp to 500 bp, or about 80 bp to 400 bp, or about 100 bp to 1 kb), although fragments having a median size outside of this range may be present. This term is intended to encompass free DNA molecules that are circulating in the bloodstream as well as DNA molecules that are present in extra-cellular vesicles (such as exosomes) that are circulating in the bloodstream.

As used herein, “foreign biological material” refers to any biological material that is not native to a host.

As used herein, “cellular origin” refers to the cell or cell-type from which a material, such as DNA, originates.

As used herein, “source origin” refers to the individual from which a material, such as DNA, originates. For example, the source origin may be a donor, or may be the host subject.

“Methylation pattern” refers to the pattern generated by the presence of methylated CpGs or non-methylated CpGs in a segment of DNA. For example, in a segment of DNA containing three CpGs. one methylation pattern is all three CpGs being methylated; a different methylation pattern is all three CpGs not being methylated; another methylation pattern is only the first CpG being methylated; yet another methylation pattern is only the second CpG being methylated; yet a different methylation pattern is the first and second CpG being methylated, etc.

“Methylation status” refers to whether a CpG dinucleotide is methylated or not methylated.

As used herein, “hypermethylated” refers to the presence of methylated CpGs. For example, a hypermethylated genomic region means that each CpG in the genomic region is methylated.

As used herein, “hypomethylated” refers to the presence of CpGs that are not methylated. For example, a hypomethylated genomic region means that each CpG in the genomic region is not methylated.

The term “sequencing” as used herein refers to a method by which the identity of at least 10 consecutive nucleotides for example, the identity of at least 20, at least 50, at least 100 or at least 200 or more consecutive nucleotides) of a polynucleotide is obtained.

The term “next-generation sequencing” as used herein refers to the parallelized sequencing-by-synthesis or sequencing-by-ligation platforms currently employed by Illumina, Life Technologies, and Roche, etc. Next-generation sequencing methods may also include nanopore sequencing methods such as that commercialized by Oxford Nanopore Technologies, electronic-detection based methods such as Ion Torrent technology commercialized by Life Technologies. or single-molecule fluorescence-based methods such as that commercialized by Pacific Biosciences.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of an active agent is an amount sufficient to carry out a specifically stated purpose.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

### Methods of the Invention

A novel method was developed to identify and quantify tissue-specific cell death from cfDNA through epigenetic markers and capture genetic information to identify cell origin from transplanted or host biological material, e.g., organs, tissues, or cells.

The use of the combination of epigenetic and genetic markers can show the detection and distinction of host-and donor-derived cells. Genetic differences between donor and host can be used to identify donor-derived cfDNA (dd-cfDNA) molecules with origins in the tissue from the donor. The dd-cfDNA molecules that also exhibit cell-specific methylation patterns can then be used to track the fate of these donor-derived cells.

Therefore the present invention relates to, in a subject receiving foreign biological material from a donor, a method of detecting donor and/or host cell death. The method comprises sequencing efDNA in a biospecimen from the subject; determining cellular origin of the cfDNA by identifying methylation patterns in the sequence of the cfDNA and comparing the methylation patterns in the sequence of the cfDNA to known methylation patterns associated with different cell types; and determining source origin of the cfDNA by genotyping the cfDNA and identifying whether the cfDNA originates from the foreign biological material or from tissue of the subject. Donor cell death can be detected when the cfDNA has (i) a cellular origin associated with the type of foreign biological material that was received from the donor, and (ii) a source origin of the donor.

The biospecimen may be a biological fluid obtained from the subject, including, but not limited to, whole blood, plasma, serum, urine, or any other fluid sample produced by the subject such as saliva, cerebrospinal fluid, urine, or sputum. In certain embodiments, the biospecimen is whole blood, plasma, or serum.

CfDNA can be obtained by centrifuging the biological fluid, such as whole blood, to remove all cells, and then isolating the DNA from the remaining plasma or serum. Such methods are well known (see, e.g., Lo et al., 1998). Circulating cfDNA can be double-stranded or single-stranded DNA.

The foreign biological material may be any biological material that comprises cells that have DNA, and that can be transplanted from a donor to a host. The cells may be organized as an organ or portion thereof, a tissue, or a population of individual cells (not organized as an organ or tissue). The population of cells may be a population of the same cell type, or a population of different cell types.

In some embodiments, the foreign biological material comprises a tissue. Examples include, but are not limited to, liver tissue, cardiac tissue, vascular tissue, pancreatic tissue, splenic tissue, esophageal tissue, gastric tissue, intestinal tissue, colon tissue, lung tissue, tracheal tissue, skin tissue, subcutaneous tissue, hair tissue, kidney tissue, connective tissue, muscular tissue, skeletal tissue, cartilage tissue, prostate tissue, bladder tissue, gonadal tissue, uterine tissue, penile tissue, neural tissue, corneal tissue, ophthalmologic tissue, bone marrow tissue

In some embodiments, the foreign biological material may comprise an organ or portion thereof, examples include, but are not limited to, liver, heart, blood vessel, pancreas, colon, lung, skin, kidney, bone, muscle, and prostate.

In some embodiments, the foreign biological material may comprise a population of cells, for instance, a population of blood-derived cells. Examples of blood-derived cells include, but are not limited to, granulocytes, natural killer cells, naïve B-cells, mature B-cells, mature T-cells, monocytes, and macrophages.

Table 1 provides examples of cellular origins associated with different types of tissue.

TABLE 1
Cellular origins, and the different types of
tissue with which they can be associated.

| Cellular Origins | Tissue |
|---|---|
| Mature B-Cell | Blood, Bone Marrow |
| Naïve B-Cell | Blood, Bone Marrow |
| Biliary Epithelial Cell | Liver |
| Breast Basal Cell | Breast |
| Breast Luminal Cell | Breast |
| Bulk Endothelial Cell | Blood Vessels |
| Bulk Epithelial Cell | Any Epithelia |
| Bulk Immune Cell | Immune Organ |
| Cardiomyocyte | Heart |
| Cardiopulmonary Endothelial Cell | Heart, Lung |
| Colon Epithelial Cell | Colon |
| Dermal Epithelial Cell | Skin |
| Granulocyte | Blood, Bone Marrow |
| Hepatocyte | Liver |
| Keratinocyte | Skin |
| Kidney Epithelial Cell | Kidney |
| Liver Endothelial Cell | Liver |
| Liver Stromal Cell | Liver |
| Liver Resident Immune Cell | Liver |
| Lung Epithelial Cell | Lung |
| Megakaryocyte | Bone Marrow |
| Monocytes and Macrophage | Blood |
| Neuron | Neural |
| Natural Killer Cell | Blood |
| Pancreatic Cell | Pancreas |
| Prostate Epithelial Cell | Prostate |
| Skeletal Muscular Cell | Skeletal Muscle |
| Mature T-Cell | Blood |

In some embodiments, the cell type identified may be indicative of particular conditions associated with the cell damage. For instance, when the foreign biological material is liver or liver tissue, detection of biliary epithelial cells may indicate biliary complications from cholangiocyte; detection of liver endothelial cells may indicate antibody-mediated rejection or graft-versus-host disease; and detection of parenchymal cells may indicate acute cellular rejection from hepatocellular. Thus, the present invention includes methods of detecting, and methods of treating, any of these liver conditions/ailments. The methods may comprise determining cellular origin of the cell-free DNA in accordance with embodiments of the invention, wherein the liver condition or ailment is detected, and in some embodiments treatment is administered, when the cell-free DNA has a cellular origin of the cell-type discussed herein.

In some embodiments, the methods of detecting cell death of the present invention may further comprise quantifying the cfDNA that is determined to have a cellular origin associated with the type of foreign biological material that was received from the donor and a source origin of the donor. Methods for quantifying the cfDNA are known in the art and include, but are not limited to, PCR; fluorescence-based quantification methods (e.g., Qubit); chromatography techniques such as gas chromatography, supercritical fluid chromatography, and liquid chromatography, such as partition chromatography, adsorption chromatography, ion exchange chromatography, size exclusion chromatography, thin-layer chromatography, and affinity chromatography; electrophoresis techniques, such as capillary electrophoresis, capillary zone electrophoresis, capillary isoelectric focusing, capillary electrochromatography, micellar electrokinetic capillary chromatography, isotachophoresis, transient isotachophoresis, and capillary gel electrophoresis; comparative genomic hybridization; microarrays; and bead arrays.

In some embodiments, the methods of detecting cell death of the present invention may be performed at timepoints relevant to tissue regeneration and/or repair, or relevant to tissue disease/dysfunction.

The present invention also relates to a method of monitoring a subject's response to receiving foreign biological material from a donor. The method comprises detecting cell death in the subject at one or more time points after receiving the foreign biological material. In some embodiments, the method further comprises quantifying cell death at multiple time points after receiving the foreign biological material.

In addition, the present invention relates to a method of treating donor cell death in a subject receiving foreign biological material from a donor. In some embodiments, the method comprises administering a treatment to the subject when donor cell death is detected. Detection of donor cell death may be in accordance with the methods of the present invention.

In some embodiments, the method of treating donor cell death in a subject receiving foreign biological material from a donor comprises administering a treatment to the subject when there is an increase in donor cell death between two or more time points after the subject receives the foreign biological material. Detection and quantification of donor cell death at two or more time points after receiving the foreign biological material may be in accordance with methods of the present invention.

Further, the present invention relates to a method of treating graft dysfunction or rejection in a subject receiving the graft from a donor. The method comprises administering a treatment to the subject when the quantity of donor cell death is increased between the time points. Detection and quantification of donor cell death at two or more time points after receiving the foreign biological material may be in accordance with methods of the present invention. In some embodiments, the graft is a foreign biological material as described herein.

The time points may be, for instance, one or more days between and including Day 0 (day of receiving the foreign biological material) through Day 60, such as Day 0, Day 1, Day 2, Day 3, Day 4, Day 5, Day 6, Day 7, Day 8 Day 9, Day 10, Day 11, Day 12, Day 13, Day 14, Day 15, Day 16, Day 17, Day 18 Day 19, Day 20, Day 21, Day 25, Day 28, Day 30, Day 35, Day 40, Day 42, Day 45, Day 49, Day 50, Day 55, Day 56, or Day 60. In certain embodiments, the time points are Day 7 and Day 30 after receiving the foreign biological material. In certain embodiments, the time points may be, or may include, a time later than Day 60 in which the subject exhibits symptoms of tissue dysfunction. In certain embodiments, the subject may be monitored at time points later than Day 60.

The increase in the quantity of cell death may be, for example, a percent increase of about 0.1% to 100%, such as about 0.1%, 0.5%, 1%, 2%, 3%, 4%, 5%, 6%, 7%, 8%, 9%, 10%, 20%, 30%, 40%, 50%, 60%, 70%, 80%, 90%, or 100%; or may be a fold increase of at least about 2-fold, such as about 2-fold, or 3-fold, or 4-fold, or 5-fold, or 6-fold, or 7-fold, or 8-fold, or 9-fold, or 10-fold. In some embodiments, the increase in the quantity of cell death may be any increase that is determined to be statistically significant (e.g., p≤0.05. p≤0.01, etc.) as calculated by statistical methods known in the art.

In some embodiments, the presence of donor cell death at time point Day 7 and Day 30 may be above threshold level of damage. Presence of any donor cell death at relevant time points and from relevant cellular origins can indicate dysfunction.

In some embodiments, the treatment may comprise an effective amount of an immunosuppressive agent, such as a corticosteroid, janus kinase inhibitor, calcineurin inhibitor, mTOR inhibitor, inosine-5′-monophosphate dehydrogenase (IMPDH) inhibitor, biologic (e.g., abatacept, adalimumab, anakinra, certolizumab, etanercept, golimumab, infliximab, ixekizumab, natalizumab, rituximab, secukinumab, tocilizumab, ustekinumab, vedolizumab), or monoclonal antibody (e.g., basilivimab, daclizumab).

In some embodiments, the treatment may comprise an anti-inflammatory agent, such as a steroidal anti-inflammatory agent or a non-steroidal anti-inflammatory agent. Examples of non-steroidal anti-inflammatory agent include, but not limited to, ketorolac, diclofenac, naproxen, meloxicam, esomeprazole, misoprostol, ibuprofen, famotidine, nabumetone, indomethacin, mefenamic acid, etodolac, piroxicam, sulindac, ketoprofen, diflunisal, oxaprozin, flurbiprofen, tolmetin, and nabumetone.

In some embodiments, the treatment may comprise an effective amount of an antibacterial or antiviral therapy.

In some embodiments, the treatment my comprise a therapy targeted to a pathway that controls cell death.

### Determination of Cellular Origin

Determination of the cellular origin of the cfDNA comprises identifying methylation patterns in the sequence of the cfDNA and comparing the methylation patterns in the sequence of the cfDNA to known methylation patterns associated with different cell types.

Different DNA methylation detection technologies may be used in the present invention. Examples include, but are not limited to, a restriction enzyme digestion approach, which involves cleaving DNA at enzyme-specific CpG sites; an affinity-enrichment method, for instance, methylated DNA immunoprecipitation sequencing (MeDIP-seq) or methyl-CpG-binding domain sequencing (MBD-seq); bisulfite conversion methods such as whole genome bisulfite sequencing (WGBS), reduced representation bisulfite sequencing (RRBS), methylated CpG tandem amplification and sequencing (MCTA-seq), and methylation arrays; enzymatic approaches, such as enzymatic methyl-sequencing (EM-seq) or ten-eleven translocation (TET)—assisted pyridine borane sequencing (TAPS); and other methods that do not require treatment of DNA, for instance, by nanopore-sequencing from Oxford Nanopore Technologies (ONT) and single molecule real-time (SMRT) sequencing from Pacific Biosciences (PacBio).

Comparison of the methylation pattern in sequence of the cfDNA with known methylation patterns may comprise identifying the presence of a methylation pattern in the sequence of the cfDNA, or a portion thereof, that are attributed to specific cell types. In some embodiments, the presence of a methylation pattern was performed by hybridization capture sequencing of cfDNA. In other embodiments, the presence of a methylation pattern was performed using bisulfite amplicon-sequencing.

The methylation pattern may comprise a segment of nucleotide sequence containing at least 1 CpG dinucleotide, or at least about 2 CpG dinucleotides, or at least about 3 CpG dinucleotides. In some embodiments, the methylation pattern may comprise a segment of nucleotide sequence containing at least about 4 CpG dinucleotides, or at least about 5 CpG dinucleotides, or at least about 6 CpG dinucleotides, or at least about 7 CpG dinucleotides, or at least about 8 CpG dinucleotides, or at least about 9 CpG dinucleotides, or at least about 10 CpG dinucleotides.

Table 2 provides methylation status at CpG dinucleotides in genomic regions that indicative of different cell types. The presence of a same methylation pattern between the sequence of the cfDNA and the genomic regions set forth in Table 2 indicates the cell-type from which the cfDNA originates. Table 2 provides contiguous methylation status across multiple adjacent CpG sites (patterns) within genomic region.

TABLE 2
Methylation status in genomic regions that are indicative of cell type.

| | | | | Methylation |
|---|---|---|---|---|
| Cell Type | Chromosome | Start* | End* | Status |
| Mature B | chr11 | 68139032 | 68139146 | Hypomethylated |
| Mature B | chr17 | 80829337 | 80829647 | Hypomethylated |
| Mature B | chr6 | 167506945 | 167507168 | Hypomethylated |
| Mature B | chr19 | 1648937 | 1649129 | Hypomethylated |
| Mature B | chr3 | 9694444 | 9695149 | Hypomethylated |
| Mature B | chr18 | 77116085 | 77116618 | Hypomethylated |
| Mature B | chr9 | 135763441 | 135764023 | Hypomethylated |
| Mature B | chr12 | 121686411 | 121686789 | Hypomethylated |
| Mature B | chr6 | 16306332 | 16306681 | Hypomethylated |
| Mature B | chr14 | 96179945 | 96180308 | Hypomethylated |
| Mature B | chr6 | 16306086 | 16306267 | Hypomethylated |
| Mature B | chr16 | 28944140 | 28944468 | Hypomethylated |
| Mature B | chr17 | 73316011 | 73316779 | Hypomethylated |
| Mature B | chr2 | 112917111 | 112917496 | Hypomethylated |
| Mature B | chr7 | 637567 | 637692 | Hypomethylated |
| Mature B | chr17 | 79233329 | 79233604 | Hypomethylated |
| Mature B | chr2 | 240291097 | 240291331 | Hypomethylated |
| Mature B | chr17 | 3493609 | 3493935 | Hypomethylated |
| Mature B | chr11 | 2415602 | 2415708 | Hypomethylated |
| Mature B | chr13 | 111329306 | 111329450 | Hypomethylated |
| Mature B | chr6 | 167507176 | 167507296 | Hypomethylated |
| Mature B | chr10 | 1704918 | 1705006 | Hypomethylated |
| Mature B | chr14 | 104158553 | 104158779 | Hypomethylated |
| Mature B | chr17 | 80873275 | 80873776 | Hypomethylated |
| Mature B | chr16 | 89180214 | 89180662 | Hypomethylated |
| Mature B | chr11 | 64567087 | 64567240 | Hypomethylated |
| Mature B | chr19 | 2324328 | 2324440 | Hypomethylated |
| Mature B | chr22 | 50196818 | 50196977 | Hypomethylated |
| Mature B | chr16 | 88103031 | 88103137 | Hypomethylated |
| Mature B | chr19 | 1621010 | 1621318 | Hypomethylated |
| Mature B | chr1 | 11395635 | 11395863 | Hypomethylated |
| Mature B | chr2 | 464998 | 465071 | Hypomethylated |
| Mature B | chr16 | 1495324 | 1495725 | Hypomethylated |
| Mature B | chr9 | 101754799 | 101755349 | Hypomethylated |
| Mature B | chr5 | 177544974 | 177545232 | Hypomethylated |
| Mature B | chr6 | 159639094 | 159639294 | Hypomethylated |
| Mature B | chr8 | 23083219 | 23083470 | Hypomethylated |
| Mature B | chr14 | 104852775 | 104853229 | Hypomethylated |
| Mature B | chr7 | 2140017 | 2140179 | Hypomethylated |
| Mature B | chr19 | 56156225 | 56156453 | Hypomethylated |
| Mature B | chr10 | 121201654 | 121201754 | Hypomethylated |
| Mature B | chr20 | 57033426 | 57033735 | Hypomethylated |
| Mature B | chr2 | 61199859 | 61200489 | Hypomethylated |
| Mature B | chr15 | 75146404 | 75146784 | Hypomethylated |
| Mature B | chr16 | 85289643 | 85290003 | Hypomethylated |
| Mature B | chr16 | 88765052 | 88765399 | Hypomethylated |
| Mature B | chr7 | 2140231 | 2140348 | Hypomethylated |
| Mature B | chr16 | 773800 | 773999 | Hypomethylated |
| Mature B | chr10 | 13330436 | 13330842 | Hypomethylated |
| Mature B | chr15 | 74714681 | 74715017 | Hypomethylated |
| Naïve B | chr11 | 68139032 | 68139146 | Hypomethylated |
| Naïve B | chr17 | 3493609 | 3493935 | Hypomethylated |
| Naïve B | chr19 | 1648937 | 1649129 | Hypomethylated |
| Naïve B | chr17 | 80829337 | 80829647 | Hypomethylated |
| Naïve B | chr22 | 20760823 | 20761115 | Hypomethylated |
| Naïve B | chr17 | 80873275 | 80873776 | Hypomethylated |
| Naïve B | chr2 | 240291097 | 240291331 | Hypomethylated |
| Naïve B | chr10 | 6262461 | 6262727 | Hypomethylated |
| Naïve B | chr11 | 64567087 | 64567240 | Hypomethylated |
| Naïve B | chr14 | 104158553 | 104158779 | Hypomethylated |
| Naïve B | chr19 | 2324328 | 2324440 | Hypomethylated |
| Naïve B | chr16 | 773800 | 773999 | Hypomethylated |
| Naïve B | chr8 | 16617187 | 16617280 | Hypomethylated |
| Naïve B | chr16 | 88103031 | 88103137 | Hypomethylated |
| Naïve B | chr5 | 177544974 | 177545232 | Hypomethylated |
| Naïve B | chr14 | 96179945 | 96180308 | Hypomethylated |
| Naïve B | chr4 | 185876236 | 185876334 | Hypomethylated |
| Naïve B | chr9 | 135763441 | 135764023 | Hypomethylated |
| Naïve B | chr11 | 2415602 | 2415708 | Hypomethylated |
| Naïve B | chr10 | 121201654 | 121201754 | Hypomethylated |
| Naïve B | chr7 | 2140017 | 2140179 | Hypomethylated |
| Naïve B | chr16 | 2516952 | 2517020 | Hypomethylated |
| Naïve B | chr16 | 75107515 | 75107841 | Hypomethylated |
| Biliary Epithelial | chr10 | 7744476 | 7744775 | Hypomethylated |
| Biliary Epithelial | chr19 | 35534546 | 35535085 | Hypomethylated |
| Biliary Epithelial | chr22 | 39685694 | 39685809 | Hypomethylated |
| Biliary Epithelial | chr16 | 87824936 | 87825139 | Hypomethylated |
| Biliary Epithelial | chr17 | 48626642 | 48627290 | Hypomethylated |
| Biliary Epithelial | chr1 | 19600102 | 19600251 | Hypomethylated |
| Biliary Epithelial | chr3 | 128141404 | 128141587 | Hypomethylated |
| Biliary Epithelial | chr5 | 53223677 | 53224181 | Hypomethylated |
| Biliary Epithelial | chr9 | 137331241 | 137331537 | Hypomethylated |
| Biliary Epithelial | chr11 | 47471307 | 47471401 | Hypomethylated |
| Biliary Epithelial | chr17 | 79042980 | 79043169 | Hypomethylated |
| Biliary Epithelial | chr22 | 37419795 | 37420227 | Hypomethylated |
| Biliary Epithelial | chr2 | 74731371 | 74731414 | Hypomethylated |
| Biliary Epithelial | chr5 | 170876495 | 170876741 | Hypomethylated |
| Biliary Epithelial | chr10 | 135340893 | 135341026 | Hypomethylated |
| Biliary Epithelial | chr20 | 56287198 | 56287355 | Hypomethylated |
| Biliary Epithelial | chr1 | 9324077 | 9324214 | Hypomethylated |
| Biliary Epithelial | chr2 | 241827906 | 241828206 | Hypomethylated |
| Biliary Epithelial | chr14 | 101944626 | 101944805 | Hypomethylated |
| Biliary Epithelial | chr21 | 46893085 | 46893254 | Hypomethylated |
| Biliary Epithelial | chr2 | 241949229 | 241949839 | Hypermethylated |
| Biliary Epithelial | chr17 | 42084940 | 42085300 | Hypermethylated |
| Biliary Epithelial | chr19 | 50095791 | 50096912 | Hypermethylated |
| Biliary Epithelial | chr10 | 11726866 | 11727466 | Hypermethylated |
| Biliary Epithelial | chr3 | 13114628 | 13114763 | Hypermethylated |
| Biliary Epithelial | chr10 | 102882978 | 102883606 | Hypermethylated |
| Biliary Epithelial | chr11 | 2160875 | 2161446 | Hypermethylated |
| Biliary Epithelial | chr19 | 3434917 | 3435465 | Hypermethylated |
| Biliary Epithelial | chr8 | 11565610 | 11565963 | Hypermethylated |
| Biliary Epithelial | chr8 | 11566699 | 11566916 | Hypermethylated |
| Biliary Epithelial | chr11 | 10328197 | 10328509 | Hypermethylated |
| Biliary Epithelial | chr8 | 11565188 | 11565531 | Hypermethylated |
| Biliary Epithelial | chr14 | 38067833 | 38067990 | Hypermethylated |
| Biliary Epithelial | chr16 | 88716968 | 88717851 | Hypermethylated |
| Biliary Epithelial | chr22 | 37464939 | 37465280 | Hypermethylated |
| Biliary Epithelial | chr7 | 44185404 | 44185968 | Hypermethylated |
| Biliary Epithelial | chr16 | 127503 | 127698 | Hypermethylated |
| Biliary Epithelial | chr3 | 197281447 | 197283152 | Hypermethylated |
| Biliary Epithelial | chr18 | 55107463 | 55107887 | Hypermethylated |
| Biliary Epithelial | chr15 | 53087579 | 53087929 | Hypermethylated |
| Biliary Epithelial | chr11 | 2161456 | 2162090 | Hypermethylated |
| Biliary Epithelial | chr14 | 38067054 | 38067566 | Hypermethylated |
| Biliary Epithelial | chr7 | 155597344 | 155597625 | Hypermethylated |
| Biliary Epithelial | chr2 | 128181286 | 128181430 | Hypermethylated |
| Biliary Epithelial | chr3 | 55515070 | 55515736 | Hypermethylated |
| Biliary Epithelial | chr6 | 152128957 | 152129855 | Hypermethylated |
| Biliary Epithelial | chr5 | 176829535 | 176830185 | Hypermethylated |
| Biliary Epithelial | chr2 | 128180566 | 128181267 | Hypermethylated |
| Biliary Epithelial | chr11 | 2164937 | 2165828 | Hypermethylated |
| Biliary Epithelial | chr8 | 27182852 | 27183553 | Hypermethylated |
| Breast Basal | chr2 | 44497562 | 44497807 | Hypomethylated |
| Breast Basal | chr17 | 4453891 | 4454402 | Hypomethylated |
| Breast Basal | chr11 | 57558973 | 57559168 | Hypomethylated |
| Breast Basal | chr11 | 392133 | 392695 | Hypomethylated |
| Breast Basal | chr22 | 47023757 | 47023904 | Hypomethylated |
| Breast Basal | chr2 | 240040057 | 240040254 | Hypomethylated |
| Breast Basal | chr1 | 3321982 | 3322140 | Hypomethylated |
| Breast Basal | chr18 | 77635864 | 77636069 | Hypomethylated |
| Breast Basal | chr22 | 40417354 | 40417567 | Hypomethylated |
| Breast Basal | chr5 | 137803144 | 137803521 | Hypomethylated |
| Breast Basal | chr11 | 391681 | 392015 | Hypomethylated |
| Breast Basal | chr16 | 27781217 | 27781639 | Hypomethylated |
| Breast Basal | chr17 | 2278727 | 2279131 | Hypomethylated |
| Breast Basal | chr17 | 70053558 | 70053834 | Hypomethylated |
| Breast Basal | chr10 | 123738139 | 123738614 | Hypomethylated |
| Breast Basal | chr16 | 81533203 | 81533549 | Hypomethylated |
| Breast Basal | chr18 | 10588980 | 10589063 | Hypomethylated |
| Breast Basal | chr18 | 10589150 | 10589279 | Hypomethylated |
| Breast Basal | chr19 | 17437804 | 17438236 | Hypomethylated |
| Breast Basal | chr19 | 46388006 | 46388262 | Hypomethylated |
| Breast Basal | chr20 | 62045021 | 62045251 | Hypomethylated |
| Breast Basal | chr1 | 2832006 | 2832144 | Hypomethylated |
| Breast Basal | chr16 | 88166706 | 88166848 | Hypomethylated |
| Breast Basal | chr2 | 233198590 | 233198791 | Hypomethylated |
| Breast Basal | chr7 | 4804631 | 4805136 | Hypomethylated |
| Breast Basal | chr8 | 142235391 | 142235926 | Hypomethylated |
| Breast Basal | chr11 | 772689 | 773090 | Hypomethylated |
| Breast Basal | chr19 | 8554999 | 8555062 | Hypomethylated |
| Breast Basal | chr19 | 46387679 | 46387919 | Hypomethylated |
| Breast Basal | chr22 | 29659845 | 29660202 | Hypomethylated |
| Breast Basal | chr14 | 102093931 | 102094226 | Hypomethylated |
| Breast Basal | chr17 | 5983894 | 5984066 | Hypomethylated |
| Breast Basal | chr22 | 47022433 | 47022659 | Hypomethylated |
| Breast Basal | chr7 | 2054500 | 2055180 | Hypomethylated |
| Breast Basal | chr11 | 2170373 | 2170444 | Hypomethylated |
| Breast Basal | chr5 | 1876857 | 1877139 | Hypermethylated |
| Breast Basal | chr10 | 8089332 | 8089925 | Hypermethylated |
| Breast Basal | chr16 | 56703476 | 56703914 | Hypermethylated |
| Breast Basal | chr9 | 135463966 | 135464285 | Hypermethylated |
| Breast Basal | chr10 | 129534178 | 129534481 | Hypermethylated |
| Breast Basal | chr13 | 37004787 | 37005108 | Hypermethylated |
| Breast Basal | chr14 | 95233855 | 95234127 | Hypermethylated |
| Breast Basal | chr6 | 10381523 | 10382075 | Hypermethylated |
| Breast Basal | chr9 | 129372570 | 129372906 | Hypermethylated |
| Breast Basal | chr12 | 49390678 | 49391209 | Hypermethylated |
| Breast Basal | chr1 | 22668552 | 22668874 | Hypermethylated |
| Breast Basal | chr3 | 137489017 | 137489723 | Hypermethylated |
| Breast Basal | chr5 | 1874836 | 1875551 | Hypermethylated |
| Breast Basal | chr12 | 54090151 | 54090388 | Hypermethylated |
| Breast Basal | chr9 | 129372913 | 129373070 | Hypermethylated |
| Breast Luminal | chr11 | 65582724 | 65582909 | Hypomethylated |
| Breast Luminal | chr1 | 3321982 | 3322140 | Hypomethylated |
| Breast Luminal | chr1 | 2832006 | 2832144 | Hypomethylated |
| Breast Luminal | chr5 | 148958721 | 148958920 | Hypomethylated |
| Breast Luminal | chr17 | 2278727 | 2279131 | Hypomethylated |
| Breast Luminal | chr5 | 176764116 | 176764365 | Hypomethylated |
| Breast Luminal | chr12 | 115150184 | 115150549 | Hypomethylated |
| Breast Luminal | chr16 | 90068365 | 90068490 | Hypomethylated |
| Breast Luminal | chr17 | 47467293 | 47467337 | Hypomethylated |
| Breast Luminal | chr18 | 10589150 | 10589279 | Hypomethylated |
| Breast Luminal | chr19 | 48491917 | 48492032 | Hypomethylated |
| Breast Luminal | chr6 | 169641078 | 169641392 | Hypomethylated |
| Breast Luminal | chr2 | 121036351 | 121037152 | Hypomethylated |
| Breast Luminal | chr16 | 30852669 | 30852999 | Hypomethylated |
| Breast Luminal | chr18 | 10588980 | 10589063 | Hypomethylated |
| Breast Luminal | chr19 | 7685239 | 7685435 | Hypomethylated |
| Breast Luminal | chr4 | 757194 | 757416 | Hypomethylated |
| Breast Luminal | chr1 | 24191684 | 24192034 | Hypomethylated |
| Breast Luminal | chr6 | 168797654 | 168797892 | Hypomethylated |
| Breast Luminal | chr9 | 139698878 | 139699275 | Hypomethylated |
| Breast Luminal | chr1 | 9565292 | 9565531 | Hypomethylated |
| Breast Luminal | chr13 | 103454025 | 103454177 | Hypomethylated |
| Breast Luminal | chr3 | 133175047 | 133175550 | Hypomethylated |
| Breast Luminal | chr1 | 3017607 | 3017703 | Hypomethylated |
| Breast Luminal | chr1 | 3474045 | 3474246 | Hypomethylated |
| Breast Luminal | chr5 | 172982435 | 172982535 | Hypomethylated |
| Breast Luminal | chr14 | 105181998 | 105182486 | Hypomethylated |
| Breast Luminal | chr14 | 105269038 | 105269404 | Hypomethylated |
| Breast Luminal | chr3 | 137489017 | 137489723 | Hypermethylated |
| Breast Luminal | chr10 | 22542284 | 22542463 | Hypermethylated |
| Breast Luminal | chr17 | 42287754 | 42288090 | Hypermethylated |
| Breast Luminal | chr5 | 138729659 | 138729816 | Hypermethylated |
| Breast Luminal | chr5 | 174158786 | 174158926 | Hypermethylated |
| Breast Luminal | chr6 | 10393138 | 10393779 | Hypermethylated |
| Breast Luminal | chr9 | 129386162 | 129386326 | Hypermethylated |
| Breast Luminal | chr5 | 174158548 | 174158782 | Hypermethylated |
| Breast Luminal | chr9 | 129372460 | 129372565 | Hypermethylated |
| Breast Luminal | chr10 | 8085311 | 8085801 | Hypermethylated |
| Breast Luminal | chr5 | 1876857 | 1877139 | Hypermethylated |
| Breast Luminal | chr5 | 1879621 | 1879706 | Hypermethylated |
| Breast Luminal | chr9 | 129373594 | 129373647 | Hypermethylated |
| Breast Luminal | chr5 | 1875800 | 1875940 | Hypermethylated |
| Breast Luminal | chr5 | 1874836 | 1875551 | Hypermethylated |
| Breast Luminal | chr9 | 129388506 | 129388993 | Hypermethylated |
| Breast Luminal | chr5 | 1877986 | 1878242 | Hypermethylated |
| Breast Luminal | chr6 | 10381523 | 10382075 | Hypermethylated |
| Breast Luminal | chr9 | 129388068 | 129388495 | Hypermethylated |
| Breast Luminal | chr9 | 129372570 | 129372906 | Hypermethylated |
| Breast Luminal | chr9 | 129372913 | 129373070 | Hypermethylated |
| Bulk Endothelial | chr9 | 139406515 | 139406839 | Hypomethylated |
| Bulk Endothelial | chr6 | 1635704 | 1635851 | Hypomethylated |
| Bulk Endothelial | chr14 | 69931518 | 69931952 | Hypomethylated |
| Bulk Endothelial | chr17 | 80803660 | 80804189 | Hypomethylated |
| Bulk Endothelial | chr7 | 4746712 | 4746898 | Hypomethylated |
| Bulk Endothelial | chr12 | 52291132 | 52291323 | Hypomethylated |
| Bulk Endothelial | chr7 | 150690506 | 150691038 | Hypomethylated |
| Bulk Endothelial | chr6 | 157877066 | 157877221 | Hypomethylated |
| Bulk Endothelial | chr16 | 2220378 | 2221058 | Hypomethylated |
| Bulk Endothelial | chr7 | 736404 | 736961 | Hypomethylated |
| Bulk Endothelial | chr6 | 46889597 | 46889761 | Hypomethylated |
| Bulk Endothelial | chr17 | 1975093 | 1975641 | Hypomethylated |
| Bulk Endothelial | chr12 | 121717850 | 121717948 | Hypomethylated |
| Bulk Endothelial | chr13 | 29329007 | 29329215 | Hypomethylated |
| Bulk Endothelial | chr19 | 11707038 | 11707247 | Hypomethylated |
| Bulk Endothelial | chr6 | 167028939 | 167029195 | Hypomethylated |
| Bulk Endothelial | chr7 | 65617005 | 65617363 | Hypomethylated |
| Bulk Endothelial | chr11 | 86662732 | 86663161 | Hypomethylated |
| Bulk Endothelial | chr14 | 105796697 | 105796899 | Hypomethylated |
| Bulk Endothelial | chr1 | 3038085 | 3038257 | Hypomethylated |
| Bulk Endothelial | chr5 | 141059753 | 141060199 | Hypomethylated |
| Bulk Endothelial | chr10 | 13726446 | 13726682 | Hypomethylated |
| Bulk Endothelial | chr11 | 76290451 | 76290615 | Hypomethylated |
| Bulk Endothelial | chr16 | 1562314 | 1562661 | Hypomethylated |
| Bulk Endothelial | chr2 | 89128482 | 89128683 | Hypomethylated |
| Bulk Endothelial | chr11 | 126301685 | 126301953 | Hypomethylated |
| Bulk Endothelial | chr7 | 131314332 | 131314441 | Hypomethylated |
| Bulk Endothelial | chr9 | 138900552 | 138900629 | Hypomethylated |
| Bulk Endothelial | chr6 | 132270337 | 132270745 | Hypomethylated |
| Bulk Endothelial | chr17 | 73506140 | 73506303 | Hypomethylated |
| Bulk Endothelial | chr17 | 79170799 | 79170897 | Hypomethylated |
| Bulk Endothelial | chr4 | 1227142 | 1227401 | Hypomethylated |
| Bulk Endothelial | chr4 | 151504816 | 151505028 | Hypomethylated |
| Bulk Endothelial | chr2 | 128430937 | 128431443 | Hypomethylated |
| Bulk Endothelial | chr5 | 38466902 | 38467249 | Hypomethylated |
| Bulk Endothelial | chr9 | 35909661 | 35910091 | Hypomethylated |
| Bulk Endothelial | chr10 | 504484 | 504785 | Hypomethylated |
| Bulk Endothelial | chr7 | 2646484 | 2646629 | Hypomethylated |
| Bulk Endothelial | chr19 | 18233987 | 18234213 | Hypomethylated |
| Bulk Endothelial | chr22 | 47188803 | 47188952 | Hypomethylated |
| Bulk Endothelial | chr4 | 1227729 | 1227950 | Hypomethylated |
| Bulk Endothelial | chr17 | 15394429 | 15394554 | Hypomethylated |
| Bulk Endothelial | chr7 | 131217702 | 131217878 | Hypomethylated |
| Bulk Endothelial | chr7 | 142984797 | 142984913 | Hypomethylated |
| Bulk Endothelial | chr17 | 73509751 | 73509964 | Hypomethylated |
| Bulk Endothelial | chr9 | 137763917 | 137764041 | Hypomethylated |
| Bulk Endothelial | chr11 | 72295847 | 72296047 | Hypermethylated |
| Bulk Endothelial | chr11 | 72295460 | 72295843 | Hypermethylated |
| Bulk Endothelial | chr19 | 8398826 | 8399120 | Hypermethylated |
| Bulk Endothelial | chr8 | 10588820 | 10589153 | Hypermethylated |
| Bulk Epithelial | chr17 | 37862101 | 37862814 | Hypomethylated |
| Bulk Epithelial | chr11 | 2397153 | 2397487 | Hypomethylated |
| Bulk Epithelial | chr11 | 27490421 | 27491031 | Hypomethylated |
| Bulk Epithelial | chr7 | 985538 | 985720 | Hypomethylated |
| Bulk Epithelial | chr10 | 45406802 | 45407004 | Hypomethylated |
| Bulk Epithelial | chr6 | 168789503 | 168789732 | Hypomethylated |
| Bulk Epithelial | chr16 | 1349427 | 1349853 | Hypomethylated |
| Bulk Epithelial | chr21 | 46840048 | 46840156 | Hypomethylated |
| Bulk Epithelial | chr1 | 1099754 | 1100006 | Hypomethylated |
| Bulk Epithelial | chr10 | 101841184 | 101841419 | Hypomethylated |
| Bulk Epithelial | chr19 | 1907759 | 1907994 | Hypomethylated |
| Bulk Epithelial | chr2 | 97171293 | 97171450 | Hypomethylated |
| Bulk Epithelial | chr9 | 97803917 | 97804138 | Hypomethylated |
| Bulk Epithelial | chr11 | 128558170 | 128558419 | Hypomethylated |
| Bulk Epithelial | chr17 | 8190993 | 8191301 | Hypomethylated |
| Bulk Epithelial | chr7 | 27163145 | 27163499 | Hypomethylated |
| Bulk Epithelial | chr17 | 79949983 | 79950241 | Hypomethylated |
| Bulk Epithelial | chr16 | 27237906 | 27238021 | Hypomethylated |
| Bulk Epithelial | chr16 | 85424260 | 85424547 | Hypomethylated |
| Bulk Epithelial | chr21 | 46678570 | 46678673 | Hypomethylated |
| Bulk Epithelial | chr1 | 1216970 | 1217402 | Hypomethylated |
| Bulk Epithelial | chr11 | 65414458 | 65414631 | Hypomethylated |
| Bulk Epithelial | chr14 | 100032685 | 100032895 | Hypomethylated |
| Bulk Epithelial | chr1 | 2782910 | 2783117 | Hypomethylated |
| Bulk Epithelial | chr17 | 48179459 | 48179672 | Hypomethylated |
| Bulk Epithelial | chr5 | 1183107 | 1183283 | Hypomethylated |
| Bulk Epithelial | chr10 | 134079332 | 134079426 | Hypomethylated |
| Bulk Epithelial | chr11 | 34622158 | 34622496 | Hypomethylated |
| Bulk Epithelial | chr14 | 100621848 | 100622287 | Hypomethylated |
| Bulk Epithelial | chr16 | 27375732 | 27375974 | Hypomethylated |
| Bulk Epithelial | chr8 | 29177514 | 29177650 | Hypomethylated |
| Bulk Epithelial | chr9 | 130504037 | 130504267 | Hypomethylated |
| Bulk Epithelial | chr13 | 20805487 | 20805590 | Hypermethylated |
| Bulk Epithelial | chr15 | 101991828 | 101991977 | Hypermethylated |
| Bulk Epithelial | chr16 | 1584118 | 1584218 | Hypermethylated |
| Bulk Epithelial | chr16 | 86962660 | 86962748 | Hypermethylated |
| Bulk Epithelial | chr4 | 4765147 | 4765382 | Hypermethylated |
| Bulk Epithelial | chr19 | 11516988 | 11517209 | Hypermethylated |
| Bulk Epithelial | chr5 | 132161281 | 132161485 | Hypermethylated |
| Bulk Epithelial | chr15 | 96866665 | 96866787 | Hypermethylated |
| Bulk Epithelial | chr11 | 124750355 | 124750431 | Hypermethylated |
| Bulk Epithelial | chr15 | 96887036 | 96887138 | Hypermethylated |
| Bulk Epithelial | chr5 | 132161628 | 132161741 | Hypermethylated |
| Bulk Epithelial | chr7 | 155598820 | 155598969 | Hypermethylated |
| Bulk Epithelial | chr15 | 96885028 | 96885331 | Hypermethylated |
| Bulk Epithelial | chr8 | 8204381 | 8204920 | Hypermethylated |
| Bulk Epithelial | chr19 | 3671800 | 3672121 | Hypermethylated |
| Bulk Epithelial | chr9 | 134148465 | 134149034 | Hypermethylated |
| Bulk Epithelial | chr7 | 155599119 | 155599231 | Hypermethylated |
| Bulk Epithelial | chr12 | 124941781 | 124942249 | Hypermethylated |
| Bulk Immune | chr3 | 67706044 | 67706706 | Hypomethylated |
| Bulk Immune | chr19 | 3179171 | 3180240 | Hypomethylated |
| Bulk Immune | chr12 | 51717273 | 51718156 | Hypomethylated |
| Bulk Immune | chr9 | 27093573 | 27093899 | Hypomethylated |
| Bulk Immune | chr9 | 36458395 | 36458863 | Hypomethylated |
| Bulk Immune | chr1 | 25291486 | 25291893 | Hypomethylated |
| Bulk Immune | chr12 | 47700770 | 47701310 | Hypomethylated |
| Bulk Immune | chr17 | 76361115 | 76361254 | Hypomethylated |
| Bulk Immune | chr11 | 111093580 | 111094004 | Hypomethylated |
| Bulk Immune | chr10 | 135202560 | 135202871 | Hypomethylated |
| Bulk Immune | chr6 | 25041762 | 25042379 | Hypomethylated |
| Bulk Immune | chr16 | 29675766 | 29676065 | Hypomethylated |
| Bulk Immune | chr2 | 43397998 | 43398155 | Hypomethylated |
| Bulk Immune | chr7 | 149566913 | 149567085 | Hypomethylated |
| Bulk Immune | chr2 | 133403571 | 133403807 | Hypomethylated |
| Bulk Immune | chr8 | 145808729 | 145808892 | Hypomethylated |
| Bulk Immune | chr11 | 2321770 | 2322051 | Hypomethylated |
| Bulk Immune | chr6 | 168107235 | 168107375 | Hypomethylated |
| Bulk Immune | chr11 | 63974540 | 63974842 | Hypomethylated |
| Bulk Immune | chr19 | 5139390 | 5139647 | Hypomethylated |
| Bulk Immune | chr3 | 196367455 | 196367896 | Hypomethylated |
| Bulk Immune | chr16 | 28996021 | 28996366 | Hypomethylated |
| Bulk Immune | chr19 | 2446619 | 2446783 | Hypomethylated |
| Bulk Immune | chr9 | 123657071 | 123657231 | Hypermethylated |
| Bulk Immune | chr2 | 233251922 | 233252099 | Hypermethylated |
| Bulk Immune | chr5 | 176827023 | 176827233 | Hypermethylated |
| Bulk Immune | chr15 | 77320552 | 77320923 | Hypermethylated |
| Bulk Immune | chr2 | 54087173 | 54087424 | Hypermethylated |
| Bulk Immune | chr8 | 120685466 | 120685687 | Hypermethylated |
| Bulk Immune | chr10 | 26855881 | 26856468 | Hypermethylated |
| Bulk Immune | chr7 | 27154911 | 27155334 | Hypermethylated |
| Bulk Immune | chr11 | 63687764 | 63688071 | Hypermethylated |
| Bulk Immune | chr1 | 25257622 | 25257987 | Hypermethylated |
| Bulk Immune | chr15 | 66999862 | 66999969 | Hypermethylated |
| Bulk Immune | chr19 | 2540684 | 2541138 | Hypermethylated |
| Bulk Immune | chr5 | 148961435 | 148961719 | Hypermethylated |
| Bulk Immune | chr10 | 101282185 | 101282353 | Hypermethylated |
| Bulk Immune | chr1 | 92946803 | 92947227 | Hypermethylated |
| Bulk Immune | chr7 | 27152969 | 27153160 | Hypermethylated |
| Bulk Immune | chr10 | 101281123 | 101281332 | Hypermethylated |
| Bulk Immune | chr16 | 67682047 | 67682428 | Hypermethylated |
| Bulk Immune | chr19 | 1070772 | 1071122 | Hypermethylated |
| Bulk Immune | chr7 | 27153188 | 27153848 | Hypermethylated |
| Bulk Immune | chr12 | 107974824 | 107975402 | Hypermethylated |
| Bulk Immune | chr19 | 49841801 | 49842135 | Hypermethylated |
| Bulk Immune | chr19 | 6475589 | 6476186 | Hypermethylated |
| Bulk Immune | chr11 | 47376572 | 47377213 | Hypermethylated |
| Bulk Immune | chr1 | 45082704 | 45083125 | Hypermethylated |
| Bulk Immune | chr19 | 49842322 | 49843076 | Hypermethylated |
| Bulk Immune | chr19 | 10444874 | 10445594 | Hypermethylated |
| Cardiomyocyte | chr5 | 150028928 | 150029306 | Hypomethylated |
| Cardiomyocyte | chr22 | 26138136 | 26138600 | Hypomethylated |
| Cardiomyocyte | chr8 | 124664836 | 124665047 | Hypomethylated |
| Cardiomyocyte | chr10 | 29186554 | 29186755 | Hypomethylated |
| Cardiomyocyte | chr1 | 16341849 | 16342452 | Hypomethylated |
| Cardiomyocyte | chr13 | 113384765 | 113384930 | Hypomethylated |
| Cardiomyocyte | chr2 | 236877092 | 236877616 | Hypomethylated |
| Cardiomyocyte | chr10 | 855857 | 856183 | Hypomethylated |
| Cardiomyocyte | chr12 | 3364736 | 3365604 | Hypomethylated |
| Cardiomyocyte | chr20 | 55981966 | 55982252 | Hypomethylated |
| Cardiomyocyte | chr5 | 80529795 | 80530214 | Hypomethylated |
| Cardiomyocyte | chr19 | 1419166 | 1419762 | Hypomethylated |
| Cardiomyocyte | chr8 | 41517980 | 41518301 | Hypomethylated |
| Cardiomyocyte | chr2 | 160031535 | 160031871 | Hypomethylated |
| Cardiomyocyte | chr18 | 19780872 | 19781199 | Hypomethylated |
| Cardiomyocyte | chr1 | 45106147 | 45106400 | Hypomethylated |
| Cardiomyocyte | chr12 | 106631875 | 106632366 | Hypomethylated |
| Cardiomyocyte | chr18 | 19780465 | 19780821 | Hypomethylated |
| Cardiomyocyte | chr6 | 17032349 | 17032642 | Hypomethylated |
| Cardiomyocyte | chr11 | 78534066 | 78534252 | Hypomethylated |
| Cardiomyocyte | chr13 | 114108292 | 114108653 | Hypomethylated |
| Cardiomyocyte | chr2 | 74645622 | 74645750 | Hypomethylated |
| Cardiomyocyte | chr3 | 11606624 | 11606964 | Hypomethylated |
| Cardiomyocyte | chr8 | 141559360 | 141559711 | Hypomethylated |
| Cardiomyocyte | chr9 | 134164500 | 134164670 | Hypomethylated |
| Cardiomyocyte | chr13 | 114137827 | 114138226 | Hypomethylated |
| Cardiomyocyte | chr16 | 46781221 | 46781714 | Hypomethylated |
| Cardiomyocyte | chr20 | 60858231 | 60858540 | Hypomethylated |
| Cardiomyocyte | chr7 | 44159762 | 44160044 | Hypomethylated |
| Cardiomyocyte | chr2 | 128430937 | 128431443 | Hypomethylated |
| Cardiomyocyte | chr2 | 240879235 | 240879556 | Hypomethylated |
| Cardiomyocyte | chr3 | 18390643 | 18390959 | Hypomethylated |
| Cardiomyocyte | chr3 | 122675153 | 122675540 | Hypomethylated |
| Cardiomyocyte | chr8 | 28923946 | 28924153 | Hypomethylated |
| Cardiomyocyte | chr20 | 60861460 | 60861874 | Hypomethylated |
| Cardiomyocyte | chr7 | 43917672 | 43917891 | Hypomethylated |
| Cardiomyocyte | chr13 | 114106471 | 114106734 | Hypomethylated |
| Cardiomyocyte | chr4 | 186578455 | 186578679 | Hypomethylated |
| Cardiomyocyte | chr9 | 135929763 | 135929928 | Hypomethylated |
| Cardiomyocyte | chr15 | 90784663 | 90784846 | Hypomethylated |
| Cardiomyocyte | chr22 | 26149485 | 26150074 | Hypomethylated |
| Cardiomyocyte | chr6 | 504028 | 504466 | Hypomethylated |
| Cardiomyocyte | chr10 | 81059280 | 81059434 | Hypomethylated |
| Cardiomyocyte | chr1 | 1478629 | 1478779 | Hypomethylated |
| Cardiomyocyte | chr2 | 241533173 | 241533990 | Hypomethylated |
| Cardiomyocyte | chr6 | 6746438 | 6746730 | Hypomethylated |
| Cardiomyocyte | chr6 | 158464164 | 158464475 | Hypomethylated |
| Cardiomyocyte | chr7 | 820811 | 821315 | Hypomethylated |
| Cardiomyocyte | chr7 | 4824534 | 4824952 | Hypomethylated |
| Cardiomyocyte | chr3 | 192125874 | 192126438 | Hypermethylated |
| Cardiopulmonary | chr11 | 128698175 | 128698361 | Hypomethylated |
| Endothelial | | | | |
| Cardiopulmonary | chr7 | 150690506 | 150691038 | Hypomethylated |
| Endothelial | | | | |
| Cardiopulmonary | chr16 | 2220378 | 2221058 | Hypomethylated |
| Endothelial | | | | |
| Cardiopulmonary | chr10 | 466647 | 467242 | Hypomethylated |
| Endothelial | | | | |
| Cardiopulmonary | chr6 | 167028939 | 167029195 | Hypomethylated |
| Endothelial | | | | |
| Cardiopulmonary | chr7 | 5549534 | 5549731 | Hypomethylated |
| Endothelial | | | | |
| Cardiopulmonary | chr8 | 96572161 | 96572434 | Hypomethylated |
| Endothelial | | | | |
| Cardiopulmonary | chr9 | 139406515 | 139406839 | Hypomethylated |
| Endothelial | | | | |
| Cardiopulmonary | chr11 | 70266256 | 70266421 | Hypomethylated |
| Endothelial | | | | |
| Cardiopulmonary | chr12 | 19565936 | 19566177 | Hypomethylated |
| Endothelial | | | | |
| Cardiopulmonary | chr17 | 80803660 | 80804189 | Hypomethylated |
| Endothelial | | | | |
| Cardiopulmonary | chr6 | 1635704 | 1635851 | Hypomethylated |
| Endothelial | | | | |
| Cardiopulmonary | chr5 | 141059753 | 141060199 | Hypomethylated |
| Endothelial | | | | |
| Cardiopulmonary | chr9 | 139406957 | 139407311 | Hypomethylated |
| Endothelial | | | | |
| Cardiopulmonary | chr7 | 142984797 | 142984913 | Hypomethylated |
| Endothelial | | | | |
| Cardiopulmonary | chr11 | 134231502 | 134231622 | Hypomethylated |
| Endothelial | | | | |
| Cardiopulmonary | chr14 | 105796697 | 105796899 | Hypomethylated |
| Endothelial | | | | |
| Cardiopulmonary | chr6 | 1616682 | 1617275 | Hypomethylated |
| Endothelial | | | | |
| Cardiopulmonary | chr17 | 73506140 | 73506303 | Hypomethylated |
| Endothelial | | | | |
| Cardiopulmonary | chr15 | 83781607 | 83781804 | Hypomethylated |
| Endothelial | | | | |
| Cardiopulmonary | chr2 | 235905958 | 235906259 | Hypomethylated |
| Endothelial | | | | |
| Cardiopulmonary | chr14 | 77351761 | 77352081 | Hypomethylated |
| Endothelial | | | | |
| Cardiopulmonary | chr15 | 74637558 | 74637731 | Hypomethylated |
| Endothelial | | | | |
| Cardiopulmonary | chr9 | 139549426 | 139549603 | Hypomethylated |
| Endothelial | | | | |
| Cardiopulmonary | chr14 | 69931518 | 69931952 | Hypomethylated |
| Endothelial | | | | |
| Cardiopulmonary | chr4 | 5753985 | 5754218 | Hypomethylated |
| Endothelial | | | | |
| Cardiopulmonary | chr9 | 35909661 | 35910091 | Hypomethylated |
| Endothelial | | | | |
| Cardiopulmonary | chr16 | 8943021 | 8943199 | Hypomethylated |
| Endothelial | | | | |
| Cardiopulmonary | chr6 | 1624186 | 1624283 | Hypomethylated |
| Endothelial | | | | |
| Cardiopulmonary | chr8 | 102464327 | 102464407 | Hypomethylated |
| Endothelial | | | | |
| Cardiopulmonary | chr9 | 138900552 | 138900629 | Hypomethylated |
| Endothelial | | | | |
| Cardiopulmonary | chr12 | 121717850 | 121717948 | Hypomethylated |
| Endothelial | | | | |
| Cardiopulmonary | chr17 | 79170799 | 79170897 | Hypomethylated |
| Endothelial | | | | |
| Cardiopulmonary | chr1 | 3038085 | 3038257 | Hypomethylated |
| Endothelial | | | | |
| Cardiopulmonary | chr13 | 29329007 | 29329215 | Hypomethylated |
| Endothelial | | | | |
| Cardiopulmonary | chr19 | 474408 | 474475 | Hypomethylated |
| Endothelial | | | | |
| Cardiopulmonary | chr19 | 3765019 | 3766508 | Hypomethylated |
| Endothelial | | | | |
| Cardiopulmonary | chr2 | 128430937 | 128431443 | Hypomethylated |
| Endothelial | | | | |
| Cardiopulmonary | chr7 | 131217702 | 131217878 | Hypomethylated |
| Endothelial | | | | |
| Cardiopulmonary | chr10 | 30317521 | 30317689 | Hypomethylated |
| Endothelial | | | | |
| Cardiopulmonary | chr19 | 17374974 | 17375446 | Hypomethylated |
| Endothelial | | | | |
| Cardiopulmonary | chr2 | 237074693 | 237074856 | Hypermethylated |
| Endothelial | | | | |
| Cardiopulmonary | chr13 | 79182267 | 79182623 | Hypermethylated |
| Endothelial | | | | |
| Cardiopulmonary | chr8 | 10588820 | 10589153 | Hypermethylated |
| Endothelial | | | | |
| Cardiopulmonary | chr19 | 8398826 | 8399120 | Hypermethylated |
| Endothelial | | | | |
| Cardiopulmonary | chr16 | 58535446 | 58535596 | Hypermethylated |
| Endothelial | | | | |
| Colon Epithelial | chr2 | 97427531 | 97428080 | Hypomethylated |
| Colon Epithelial | chr13 | 114189623 | 114190065 | Hypomethylated |
| Colon Epithelial | chr17 | 80535398 | 80535834 | Hypomethylated |
| Colon Epithelial | chr7 | 150068607 | 150068986 | Hypomethylated |
| Colon Epithelial | chr13 | 30707459 | 30707773 | Hypomethylated |
| Colon Epithelial | chr6 | 38141763 | 38142021 | Hypomethylated |
| Colon Epithelial | chr19 | 10823619 | 10823914 | Hypomethylated |
| Colon Epithelial | chr2 | 106959820 | 106960122 | Hypomethylated |
| Colon Epithelial | chr20 | 55959154 | 55959798 | Hypomethylated |
| Colon Epithelial | chr17 | 76991224 | 76991699 | Hypomethylated |
| Colon Epithelial | chr1 | 1062975 | 1063187 | Hypomethylated |
| Colon Epithelial | chr12 | 132423665 | 132423879 | Hypomethylated |
| Colon Epithelial | chr14 | 104547801 | 104548104 | Hypomethylated |
| Colon Epithelial | chr9 | 140683345 | 140683528 | Hypomethylated |
| Colon Epithelial | chr11 | 66631299 | 66631470 | Hypomethylated |
| Colon Epithelial | chr16 | 2141861 | 2142285 | Hypomethylated |
| Colon Epithelial | chr17 | 5993587 | 5993793 | Hypomethylated |
| Colon Epithelial | chr17 | 77073929 | 77075062 | Hypomethylated |
| Colon Epithelial | chr17 | 79224246 | 79224909 | Hypomethylated |
| Colon Epithelial | chr9 | 130675536 | 130676110 | Hypomethylated |
| Colon Epithelial | chr8 | 142984528 | 142984693 | Hypomethylated |
| Colon Epithelial | chr17 | 63289695 | 63289933 | Hypomethylated |
| Colon Epithelial | chr19 | 2305151 | 2305259 | Hypomethylated |
| Colon Epithelial | chr1 | 6421300 | 6421678 | Hypomethylated |
| Colon Epithelial | chr5 | 664202 | 664415 | Hypomethylated |
| Colon Epithelial | chr7 | 834375 | 834637 | Hypomethylated |
| Colon Epithelial | chr7 | 157372332 | 157372448 | Hypomethylated |
| Colon Epithelial | chr11 | 1444733 | 1445067 | Hypomethylated |
| Colon Epithelial | chr11 | 68175988 | 68176320 | Hypomethylated |
| Colon Epithelial | chr16 | 87778775 | 87779046 | Hypomethylated |
| Colon Epithelial | chr14 | 93155127 | 93155269 | Hypomethylated |
| Colon Epithelial | chr19 | 2278406 | 2278613 | Hypomethylated |
| Colon Epithelial | chr1 | 1063255 | 1063374 | Hypomethylated |
| Colon Epithelial | chr6 | 35109390 | 35109799 | Hypomethylated |
| Colon Epithelial | chr17 | 79223904 | 79224188 | Hypomethylated |
| Colon Epithelial | chr7 | 959725 | 960076 | Hypomethylated |
| Colon Epithelial | chr9 | 132371085 | 132371258 | Hypomethylated |
| Colon Epithelial | chr9 | 139419864 | 139420019 | Hypomethylated |
| Colon Epithelial | chr15 | 40641488 | 40642094 | Hypomethylated |
| Colon Epithelial | chr1 | 1061483 | 1061760 | Hypomethylated |
| Colon Epithelial | chr19 | 2278728 | 2278941 | Hypomethylated |
| Colon Epithelial | chr8 | 145721522 | 145722011 | Hypomethylated |
| Colon Epithelial | chr11 | 1258312 | 1258485 | Hypomethylated |
| Colon Epithelial | chr19 | 3966588 | 3966908 | Hypomethylated |
| Colon Epithelial | chr10 | 11206756 | 11207474 | Hypermethylated |
| Colon Epithelial | chr14 | 38679781 | 38680291 | Hypermethylated |
| Colon Epithelial | chr7 | 156798471 | 156798811 | Hypermethylated |
| Colon Epithelial | chr7 | 156797298 | 156797842 | Hypermethylated |
| Colon Epithelial | chr17 | 70215747 | 70216403 | Hypermethylated |
| Colon Epithelial | chr7 | 156797845 | 156798469 | Hypermethylated |
| Dermal Endothelial | chr6 | 167028939 | 167029195 | Hypomethylated |
| Dermal Endothelial | chr17 | 80803660 | 80804189 | Hypomethylated |
| Dermal Endothelial | chr10 | 121169671 | 121170069 | Hypomethylated |
| Dermal Endothelial | chr8 | 140748972 | 140749334 | Hypomethylated |
| Dermal Endothelial | chr17 | 700763 | 701051 | Hypomethylated |
| Dermal Endothelial | chr16 | 2220378 | 2221058 | Hypomethylated |
| Dermal Endothelial | chr11 | 128698175 | 128698361 | Hypomethylated |
| Dermal Endothelial | chr11 | 134231502 | 134231622 | Hypomethylated |
| Dermal Endothelial | chr19 | 17374974 | 17375446 | Hypomethylated |
| Dermal Endothelial | chr4 | 38690557 | 38691119 | Hypomethylated |
| Dermal Endothelial | chr19 | 11707038 | 11707247 | Hypomethylated |
| Dermal Endothelial | chr7 | 150690506 | 150691038 | Hypomethylated |
| Dermal Endothelial | chr22 | 19508947 | 19509559 | Hypomethylated |
| Dermal Endothelial | chr1 | 4237464 | 4237671 | Hypomethylated |
| Dermal Endothelial | chr3 | 3079964 | 3080198 | Hypomethylated |
| Dermal Endothelial | chr17 | 5993587 | 5993793 | Hypomethylated |
| Dermal Endothelial | chr17 | 79170799 | 79170897 | Hypomethylated |
| Dermal Endothelial | chr19 | 18233987 | 18234213 | Hypomethylated |
| Dermal Endothelial | chr9 | 139406515 | 139406839 | Hypomethylated |
| Dermal Endothelial | chr2 | 109891992 | 109892086 | Hypomethylated |
| Dermal Endothelial | chr9 | 75094281 | 75094544 | Hypomethylated |
| Dermal Endothelial | chr19 | 17000794 | 17001198 | Hypomethylated |
| Dermal Endothelial | chr1 | 54110431 | 54110775 | Hypomethylated |
| Dermal Endothelial | chr2 | 43270898 | 43271814 | Hypomethylated |
| Dermal Endothelial | chr12 | 121717850 | 121717948 | Hypomethylated |
| Dermal Endothelial | chr19 | 19304585 | 19304917 | Hypomethylated |
| Dermal Endothelial | chr22 | 27026220 | 27026502 | Hypomethylated |
| Dermal Endothelial | chr1 | 7550010 | 7550091 | Hypomethylated |
| Dermal Endothelial | chr7 | 2753426 | 2753730 | Hypomethylated |
| Dermal Endothelial | chr7 | 158415936 | 158416191 | Hypomethylated |
| Dermal Endothelial | chr16 | 81731208 | 81731484 | Hypomethylated |
| Dermal Endothelial | chr5 | 179806847 | 179806982 | Hypomethylated |
| Dermal Endothelial | chr10 | 3613740 | 3613899 | Hypomethylated |
| Dermal Endothelial | chr13 | 114926731 | 114926860 | Hypomethylated |
| Dermal Endothelial | chr16 | 4815857 | 4815987 | Hypomethylated |
| Dermal Endothelial | chr17 | 79170598 | 79170789 | Hypomethylated |
| Dermal Endothelial | chr7 | 2003132 | 2003772 | Hypomethylated |
| Dermal Endothelial | chr11 | 72295460 | 72295843 | Hypermethylated |
| Dermal Endothelial | chr3 | 128209966 | 128210732 | Hypermethylated |
| Dermal Endothelial | chr11 | 128555051 | 128555481 | Hypermethylated |
| Dermal Endothelial | chr8 | 10590177 | 10590330 | Hypermethylated |
| Dermal Endothelial | chr3 | 129062831 | 129063119 | Hypermethylated |
| Dermal Endothelial | chr7 | 5468088 | 5469462 | Hypermethylated |
| Dermal Endothelial | chr2 | 177022967 | 177023205 | Hypermethylated |
| Dermal Endothelial | chr11 | 72300978 | 72301585 | Hypermethylated |
| Dermal Endothelial | chr6 | 5998982 | 5999270 | Hypermethylated |
| Dermal Endothelial | chr2 | 177022693 | 177022963 | Hypermethylated |
| Dermal Endothelial | chr2 | 177021874 | 177022246 | Hypermethylated |
| Dermal Endothelial | chr15 | 41218119 | 41218738 | Hypermethylated |
| Dermal Endothelial | chr9 | 124888894 | 124889382 | Hypermethylated |
| Granulocyte | chr14 | 102676909 | 102677377 | Hypomethylated |
| Granulocyte | chr16 | 88906304 | 88906775 | Hypomethylated |
| Granulocyte | chr19 | 1423555 | 1423830 | Hypomethylated |
| Granulocyte | chr19 | 1423535 | 1423830 | Hypomethylated |
| Granulocyte | chr13 | 114263370 | 114263522 | Hypomethylated |
| Granulocyte | chr16 | 88907037 | 88907250 | Hypomethylated |
| Granulocyte | chr17 | 4081291 | 4081574 | Hypomethylated |
| Granulocyte | chr9 | 139812189 | 139812430 | Hypomethylated |
| Granulocyte | chr17 | 694980 | 695078 | Hypomethylated |
| Granulocyte | chr4 | 89446367 | 89446620 | Hypomethylated |
| Granulocyte | chr16 | 3639093 | 3639262 | Hypomethylated |
| Granulocyte | chr9 | 129184164 | 129184294 | Hypomethylated |
| Granulocyte | chr3 | 42265452 | 42265625 | Hypomethylated |
| Granulocyte | chr10 | 119794366 | 119794600 | Hypomethylated |
| Granulocyte | chr12 | 133248726 | 133249006 | Hypomethylated |
| Granulocyte | chr16 | 8943315 | 8943550 | Hypomethylated |
| Granulocyte | chr17 | 79239868 | 79240158 | Hypomethylated |
| Granulocyte | chr1 | 1695444 | 1695532 | Hypomethylated |
| Granulocyte | chr8 | 142180100 | 142180164 | Hypomethylated |
| Granulocyte | chr2 | 209223877 | 209224762 | Hypomethylated |
| Granulocyte | chr14 | 23586796 | 23587093 | Hypomethylated |
| Granulocyte | chr16 | 85561155 | 85561229 | Hypomethylated |
| Granulocyte | chr17 | 33425704 | 33427253 | Hypomethylated |
| Granulocyte | chr17 | 78748036 | 78748234 | Hypomethylated |
| Granulocyte | chr12 | 124908476 | 124908602 | Hypomethylated |
| Granulocyte | chr15 | 101093772 | 101093974 | Hypomethylated |
| Granulocyte | chr17 | 79244235 | 79244356 | Hypomethylated |
| Granulocyte | chr2 | 120516628 | 120516725 | Hypomethylated |
| Granulocyte | chr5 | 177956161 | 177956259 | Hypomethylated |
| Granulocyte | chr10 | 73498445 | 73498902 | Hypomethylated |
| Granulocyte | chr20 | 1785056 | 1785521 | Hypomethylated |
| Granulocyte | chr9 | 136919731 | 136919892 | Hypomethylated |
| Granulocyte | chr13 | 114262862 | 114263522 | Hypomethylated |
| Granulocyte | chr20 | 62522393 | 62522519 | Hypomethylated |
| Granulocyte | chr8 | 131000173 | 131000872 | Hypomethylated |
| Hepatocyte | chr19 | 2790708 | 2791240 | Hypomethylated |
| Hepatocyte | chr2 | 118674801 | 118675049 | Hypomethylated |
| Hepatocyte | chr12 | 133249223 | 133249419 | Hypomethylated |
| Hepatocyte | chr2 | 128176259 | 128176803 | Hypomethylated |
| Hepatocyte | chr19 | 16627211 | 16627478 | Hypomethylated |
| Hepatocyte | chr16 | 27226453 | 27227400 | Hypomethylated |
| Hepatocyte | chr19 | 59022243 | 59023070 | Hypomethylated |
| Hepatocyte | chr2 | 119980528 | 119980922 | Hypomethylated |
| Hepatocyte | chr17 | 41019749 | 41020862 | Hypomethylated |
| Hepatocyte | chr2 | 44065003 | 44065200 | Hypomethylated |
| Hepatocyte | chr4 | 155507816 | 155508049 | Hypomethylated |
| Hepatocyte | chr11 | 47267183 | 47267393 | Hypomethylated |
| Hepatocyte | chr22 | 50644494 | 50644958 | Hypomethylated |
| Hepatocyte | chr14 | 70263750 | 70263921 | Hypomethylated |
| Hepatocyte | chr14 | 103573762 | 103574056 | Hypomethylated |
| Hepatocyte | chr17 | 48540012 | 48540441 | Hypomethylated |
| Hepatocyte | chr19 | 11347217 | 11347465 | Hypomethylated |
| Hepatocyte | chr20 | 60753603 | 60754165 | Hypomethylated |
| Hepatocyte | chr22 | 38212483 | 38213122 | Hypomethylated |
| Hepatocyte | chr9 | 130551728 | 130551831 | Hypomethylated |
| Hepatocyte | chr16 | 1991285 | 1991497 | Hypomethylated |
| Hepatocyte | chr17 | 80197816 | 80197957 | Hypomethylated |
| Hepatocyte | chr15 | 64996479 | 64997466 | Hypomethylated |
| Hepatocyte | chr16 | 12354976 | 12355424 | Hypomethylated |
| Hepatocyte | chr16 | 31473751 | 31474229 | Hypomethylated |
| Hepatocyte | chr17 | 80052709 | 80053033 | Hypomethylated |
| Hepatocyte | chr4 | 185724474 | 185724838 | Hypomethylated |
| Hepatocyte | chr12 | 57625174 | 57625730 | Hypomethylated |
| Hepatocyte | chr2 | 44065230 | 44065928 | Hypomethylated |
| Hepatocyte | chr2 | 127818028 | 127818420 | Hypomethylated |
| Hepatocyte | chr7 | 1912065 | 1912694 | Hypomethylated |
| Hepatocyte | chr7 | 158673837 | 158674009 | Hypomethylated |
| Hepatocyte | chr20 | 62365874 | 62366578 | Hypomethylated |
| Hepatocyte | chr1 | 11106576 | 11107083 | Hypomethylated |
| Hepatocyte | chr2 | 44066294 | 44066867 | Hypomethylated |
| Hepatocyte | chr11 | 679700 | 680254 | Hypomethylated |
| Hepatocyte | chr17 | 27493494 | 27493786 | Hypomethylated |
| Hepatocyte | chr22 | 50644200 | 50644478 | Hypomethylated |
| Hepatocyte | chr12 | 3194286 | 3194554 | Hypomethylated |
| Hepatocyte | chr16 | 72981972 | 72982166 | Hypomethylated |
| Hepatocyte | chr19 | 3659410 | 3659740 | Hypomethylated |
| Hepatocyte | chr20 | 43108645 | 43109079 | Hypomethylated |
| Hepatocyte | chr14 | 95028058 | 95028332 | Hypomethylated |
| Hepatocyte | chr9 | 139840215 | 139840491 | Hypomethylated |
| Hepatocyte | chr12 | 109639260 | 109639506 | Hypomethylated |
| Hepatocyte | chr17 | 17463355 | 17463878 | Hypomethylated |
| Hepatocyte | chr22 | 18324579 | 18324626 | Hypomethylated |
| Hepatocyte | chr3 | 126060006 | 126060381 | Hypomethylated |
| Hepatocyte | chr4 | 6755163 | 6755373 | Hypomethylated |
| Hepatocyte | chr12 | 7280736 | 7281344 | Hypomethylated |
| Keratinocyte | chr7 | 4802065 | 4802254 | Hypomethylated |
| Keratinocyte | chr8 | 143463390 | 143463895 | Hypomethylated |
| Keratinocyte | chr2 | 98349354 | 98349712 | Hypomethylated |
| Keratinocyte | chr11 | 65306835 | 65307074 | Hypomethylated |
| Keratinocyte | chr11 | 391681 | 392015 | Hypomethylated |
| Keratinocyte | chr11 | 392133 | 392695 | Hypomethylated |
| Keratinocyte | chr1 | 3321982 | 3322140 | Hypomethylated |
| Keratinocyte | chr5 | 178565867 | 178566128 | Hypomethylated |
| Keratinocyte | chr17 | 61558935 | 61559387 | Hypomethylated |
| Keratinocyte | chr16 | 3129975 | 3130351 | Hypomethylated |
| Keratinocyte | chr17 | 55037093 | 55037359 | Hypomethylated |
| Keratinocyte | chr6 | 36936452 | 36936717 | Hypomethylated |
| Keratinocyte | chr16 | 1017921 | 1018739 | Hypomethylated |
| Keratinocyte | chr20 | 62705520 | 62705726 | Hypomethylated |
| Keratinocyte | chr1 | 3322151 | 3322267 | Hypomethylated |
| Keratinocyte | chr5 | 465181 | 465615 | Hypomethylated |
| Keratinocyte | chr17 | 79244962 | 79245204 | Hypomethylated |
| Keratinocyte | chr22 | 47022433 | 47022659 | Hypomethylated |
| Keratinocyte | chr2 | 97423178 | 97423608 | Hypomethylated |
| Keratinocyte | chr8 | 143869435 | 143869513 | Hypomethylated |
| Keratinocyte | chr16 | 2334301 | 2334759 | Hypomethylated |
| Keratinocyte | chr20 | 18295551 | 18295861 | Hypomethylated |
| Keratinocyte | chr1 | 2309948 | 2310095 | Hypomethylated |
| Keratinocyte | chr9 | 94572657 | 94572869 | Hypomethylated |
| Keratinocyte | chr12 | 3190418 | 3190860 | Hypomethylated |
| Keratinocyte | chr12 | 98986220 | 98986393 | Hypomethylated |
| Keratinocyte | chr6 | 167506945 | 167507168 | Hypomethylated |
| Keratinocyte | chr20 | 18167947 | 18168360 | Hypomethylated |
| Keratinocyte | chr11 | 460362 | 460681 | Hypomethylated |
| Keratinocyte | chr17 | 76027640 | 76027890 | Hypomethylated |
| Keratinocyte | chr7 | 99227275 | 99227516 | Hypomethylated |
| Keratinocyte | chr11 | 65582724 | 65582909 | Hypomethylated |
| Keratinocyte | chr7 | 5648142 | 5648380 | Hypomethylated |
| Keratinocyte | chr8 | 102076454 | 102076803 | Hypomethylated |
| Keratinocyte | chr8 | 142235391 | 142235926 | Hypomethylated |
| Keratinocyte | chr10 | 13771352 | 13771658 | Hypomethylated |
| Keratinocyte | chr11 | 131707430 | 131707776 | Hypomethylated |
| Keratinocyte | chr9 | 79631906 | 79632226 | Hypermethylated |
| Keratinocyte | chr9 | 129386162 | 129386326 | Hypermethylated |
| Keratinocyte | chr10 | 8094621 | 8094861 | Hypermethylated |
| Keratinocyte | chr10 | 119292088 | 119292379 | Hypermethylated |
| Keratinocyte | chr12 | 54384508 | 54385130 | Hypermethylated |
| Keratinocyte | chr10 | 119292418 | 119292676 | Hypermethylated |
| Keratinocyte | chr12 | 54359358 | 54359894 | Hypermethylated |
| Keratinocyte | chr12 | 54338915 | 54339051 | Hypermethylated |
| Keratinocyte | chr10 | 119294251 | 119294603 | Hypermethylated |
| Keratinocyte | chr9 | 129372913 | 129373070 | Hypermethylated |
| Keratinocyte | chr9 | 129388068 | 129388495 | Hypermethylated |
| Keratinocyte | chr9 | 129388506 | 129388993 | Hypermethylated |
| Keratinocyte | chr12 | 54338568 | 54338887 | Hypermethylated |
| Kidney Epithelial | chr10 | 64041172 | 64041512 | Hypomethylated |
| Kidney Epithelial | chr4 | 1625248 | 1625493 | Hypomethylated |
| Kidney Epithelial | chr4 | 8642130 | 8642286 | Hypomethylated |
| Kidney Epithelial | chr4 | 42363614 | 42363736 | Hypomethylated |
| Kidney Epithelial | chr12 | 4554786 | 4554972 | Hypomethylated |
| Kidney Epithelial | chr17 | 74908539 | 74908715 | Hypomethylated |
| Kidney Epithelial | chr10 | 44163409 | 44163531 | Hypomethylated |
| Kidney Epithelial | chr22 | 39685694 | 39685809 | Hypomethylated |
| Kidney Epithelial | chr10 | 134945578 | 134945672 | Hypomethylated |
| Kidney Epithelial | chr9 | 91321534 | 91321820 | Hypomethylated |
| Kidney Epithelial | chr11 | 132582078 | 132582512 | Hypomethylated |
| Kidney Epithelial | chr11 | 132912309 | 132912375 | Hypomethylated |
| Kidney Epithelial | chr7 | 157427089 | 157427365 | Hypomethylated |
| Kidney Epithelial | chr12 | 53517541 | 53517743 | Hypomethylated |
| Kidney Epithelial | chr19 | 1961122 | 1961657 | Hypomethylated |
| Kidney Epithelial | chr5 | 176225786 | 176225897 | Hypomethylated |
| Kidney Epithelial | chr11 | 132031389 | 132031554 | Hypomethylated |
| Kidney Epithelial | chr4 | 188097977 | 188098247 | Hypomethylated |
| Kidney Epithelial | chr22 | 23607590 | 23607758 | Hypomethylated |
| Kidney Epithelial | chr12 | 132661484 | 132661640 | Hypomethylated |
| Kidney Epithelial | chr16 | 895422 | 895536 | Hypomethylated |
| Kidney Epithelial | chr10 | 504484 | 504785 | Hypomethylated |
| Kidney Epithelial | chr12 | 125242868 | 125242959 | Hypomethylated |
| Kidney Epithelial | chr17 | 75695335 | 75695465 | Hypomethylated |
| Kidney Epithelial | chr18 | 7231222 | 7232053 | Hypomethylated |
| Kidney Epithelial | chr2 | 34902628 | 34903058 | Hypomethylated |
| Kidney Epithelial | chr11 | 116484298 | 116484379 | Hypomethylated |
| Kidney Epithelial | chr18 | 76151279 | 76151406 | Hypomethylated |
| Kidney Epithelial | chr1 | 3680249 | 3680473 | Hypomethylated |
| Kidney Epithelial | chr4 | 8642296 | 8642353 | Hypomethylated |
| Kidney Epithelial | chr14 | 104834370 | 104834458 | Hypomethylated |
| Kidney Epithelial | chr4 | 619473 | 619638 | Hypomethylated |
| Kidney Epithelial | chr10 | 135120066 | 135120641 | Hypomethylated |
| Kidney Epithelial | chr4 | 1641960 | 1642062 | Hypomethylated |
| Kidney Epithelial | chr8 | 122961356 | 122961731 | Hypomethylated |
| Kidney Epithelial | chr19 | 34178390 | 34178526 | Hypomethylated |
| Kidney Epithelial | chr1 | 4193830 | 4193883 | Hypomethylated |
| Kidney Epithelial | chr4 | 100574404 | 100574537 | Hypomethylated |
| Kidney Epithelial | chr7 | 157258798 | 157258884 | Hypomethylated |
| Kidney Epithelial | chr16 | 3142948 | 3143251 | Hypomethylated |
| Kidney Epithelial | chr17 | 80192099 | 80192259 | Hypomethylated |
| Kidney Epithelial | chr19 | 10823619 | 10823914 | Hypomethylated |
| Kidney Epithelial | chr5 | 72677195 | 72677378 | Hypermethylated |
| Kidney Epithelial | chr1 | 47911646 | 47911941 | Hypermethylated |
| Kidney Epithelial | chr5 | 72597296 | 72597969 | Hypermethylated |
| Kidney Epithelial | chr5 | 72677395 | 72677689 | Hypermethylated |
| Kidney Epithelial | chr10 | 102586191 | 102586498 | Hypermethylated |
| Kidney Epithelial | chr10 | 102588392 | 102589472 | Hypermethylated |
| Kidney Epithelial | chr10 | 102586514 | 102588293 | Hypermethylated |
| Liver Endothelial | chr11 | 128698175 | 128698361 | Hypomethylated |
| Liver Endothelial | chr10 | 121169671 | 121170069 | Hypomethylated |
| Liver Endothelial | chr17 | 80803660 | 80804189 | Hypomethylated |
| Liver Endothelial | chr19 | 11707038 | 11707247 | Hypomethylated |
| Liver Endothelial | chr13 | 29329007 | 29329215 | Hypomethylated |
| Liver Endothelial | chr19 | 18233987 | 18234213 | Hypomethylated |
| Liver Endothelial | chr1 | 3038085 | 3038257 | Hypomethylated |
| Liver Endothelial | chr7 | 150690506 | 150691038 | Hypomethylated |
| Liver Endothelial | chr19 | 4983547 | 4983872 | Hypomethylated |
| Liver Endothelial | chr1 | 4237464 | 4237671 | Hypomethylated |
| Liver Endothelial | chr16 | 1562314 | 1562661 | Hypomethylated |
| Liver Endothelial | chr6 | 167028939 | 167029195 | Hypomethylated |
| Liver Endothelial | chr14 | 69931518 | 69931952 | Hypomethylated |
| Liver Endothelial | chr13 | 29328750 | 29328980 | Hypomethylated |
| Liver Endothelial | chr17 | 79170799 | 79170897 | Hypomethylated |
| Liver Endothelial | chr7 | 142984797 | 142984913 | Hypomethylated |
| Liver Endothelial | chr9 | 35909661 | 35910091 | Hypomethylated |
| Liver Endothelial | chr2 | 109891992 | 109892086 | Hypomethylated |
| Liver Endothelial | chr6 | 1635704 | 1635851 | Hypomethylated |
| Liver Endothelial | chr11 | 70266256 | 70266421 | Hypomethylated |
| Liver Endothelial | chr12 | 52291132 | 52291323 | Hypomethylated |
| Liver Endothelial | chr16 | 2220378 | 2221058 | Hypomethylated |
| Liver Endothelial | chr16 | 8943021 | 8943199 | Hypomethylated |
| Liver Endothelial | chr12 | 117113249 | 117113487 | Hypomethylated |
| Liver Endothelial | chr1 | 101705632 | 101705773 | Hypomethylated |
| Liver Endothelial | chr7 | 131217702 | 131217878 | Hypomethylated |
| Liver Endothelial | chr9 | 138900552 | 138900629 | Hypomethylated |
| Liver Endothelial | chr17 | 1975093 | 1975641 | Hypomethylated |
| Liver Endothelial | chr6 | 159549081 | 159549217 | Hypomethylated |
| Liver Endothelial | chr9 | 139406515 | 139406839 | Hypomethylated |
| Liver Endothelial | chr12 | 121717850 | 121717948 | Hypomethylated |
| Liver Endothelial | chr15 | 83781607 | 83781804 | Hypomethylated |
| Liver Endothelial | chr11 | 72300978 | 72301585 | Hypermethylated |
| Liver Endothelial | chr19 | 8398826 | 8399120 | Hypermethylated |
| Liver Stromal | chr6 | 125583698 | 125584141 | Hypomethylated |
| Liver Stromal | chr19 | 10927122 | 10928380 | Hypomethylated |
| Liver Stromal | chr8 | 96572161 | 96572434 | Hypomethylated |
| Liver Stromal | chr1 | 115826123 | 115826716 | Hypomethylated |
| Liver Stromal | chr2 | 238187070 | 238187250 | Hypomethylated |
| Liver Stromal | chr2 | 10501114 | 10501274 | Hypomethylated |
| Liver Stromal | chr15 | 67457630 | 67458134 | Hypomethylated |
| Liver Stromal | chr16 | 88121144 | 88121410 | Hypomethylated |
| Liver Stromal | chr6 | 22568625 | 22569458 | Hypomethylated |
| Liver Stromal | chr16 | 87260962 | 87261334 | Hypomethylated |
| Liver Stromal | chr6 | 109274387 | 109274515 | Hypomethylated |
| Liver Stromal | chr2 | 168614623 | 168615088 | Hypomethylated |
| Liver Stromal | chr2 | 239860016 | 239860285 | Hypomethylated |
| Liver Stromal | chr9 | 139256433 | 139256703 | Hypomethylated |
| Liver Stromal | chr17 | 48263099 | 48264061 | Hypomethylated |
| Liver Stromal | chr1 | 879443 | 879810 | Hypomethylated |
| Liver Stromal | chr7 | 1162971 | 1163186 | Hypomethylated |
| Liver Stromal | chr12 | 124514696 | 124514989 | Hypomethylated |
| Liver Stromal | chr16 | 87261416 | 87261576 | Hypomethylated |
| Liver Stromal | chr19 | 11276278 | 11277072 | Hypomethylated |
| Liver Stromal | chr20 | 48600915 | 48601197 | Hypomethylated |
| Liver Stromal | chr3 | 141162003 | 141163508 | Hypomethylated |
| Liver Stromal | chr7 | 616047 | 616267 | Hypomethylated |
| Liver Stromal | chr7 | 1953582 | 1954011 | Hypomethylated |
| Liver Stromal | chr12 | 9478964 | 9479222 | Hypomethylated |
| Liver Stromal | chr6 | 2579510 | 2579743 | Hypomethylated |
| Liver Stromal | chr10 | 131813008 | 131813140 | Hypomethylated |
| Liver Stromal | chr8 | 97166708 | 97167180 | Hypermethylated |
| Liver Stromal | chr4 | 174415351 | 174415831 | Hypermethylated |
| Liver Stromal | chr5 | 92907765 | 92907931 | Hypermethylated |
| Liver Stromal | chr19 | 48833395 | 48833967 | Hypermethylated |
| Liver Stromal | chr5 | 92908300 | 92908694 | Hypermethylated |
| Liver Stromal | chr7 | 35295220 | 35295353 | Hypermethylated |
| Liver Stromal | chr7 | 35297778 | 35298218 | Hypermethylated |
| Liver Stromal | chr15 | 37402482 | 37402724 | Hypermethylated |
| Liver Stromal | chr15 | 76634052 | 76634571 | Hypermethylated |
| Liver Stromal | chr15 | 76634581 | 76634822 | Hypermethylated |
| Liver Stromal | chr16 | 86530027 | 86530682 | Hypermethylated |
| Liver Stromal | chr7 | 35295997 | 35296479 | Hypermethylated |
| Liver Stromal | chr16 | 86537916 | 86538268 | Hypermethylated |
| Liver Stromal | chr16 | 86538276 | 86538344 | Hypermethylated |
| Liver Stromal | chr5 | 92907932 | 92908202 | Hypermethylated |
| Liver Stromal | chr5 | 122434379 | 122434629 | Hypermethylated |
| Liver Stromal | chr8 | 72917066 | 72917696 | Hypermethylated |
| Liver Stromal | chr16 | 86535785 | 86536240 | Hypermethylated |
| Liver Stromal | chr5 | 122435167 | 122435525 | Hypermethylated |
| Liver Stromal | chr16 | 86528332 | 86529004 | Hypermethylated |
| Liver Stromal | chr16 | 86529012 | 86529284 | Hypermethylated |
| Liver Stromal | chr16 | 86540831 | 86541510 | Hypermethylated |
| Liver Stromal | chr16 | 86529518 | 86529935 | Hypermethylated |
| Liver Resident | chr1 | 167485764 | 167486300 | Hypomethylated |
| Immune | | | | |
| Liver Resident | chr13 | 100004173 | 100004739 | Hypomethylated |
| Immune | | | | |
| Liver Resident | chr10 | 62671028 | 62672100 | Hypomethylated |
| Immune | | | | |
| Liver Resident | chr5 | 1172977 | 1173240 | Hypomethylated |
| Immune | | | | |
| Liver Resident | chr16 | 89408213 | 89408323 | Hypomethylated |
| Immune | | | | |
| Liver Resident | chr12 | 122711771 | 122712065 | Hypomethylated |
| Immune | | | | |
| Liver Resident | chr5 | 125798690 | 125800185 | Hypomethylated |
| Immune | | | | |
| Liver Resident | chr19 | 10226740 | 10226846 | Hypomethylated |
| Immune | | | | |
| Liver Resident | chr6 | 159457246 | 159457551 | Hypomethylated |
| Immune | | | | |
| Liver Resident | chr11 | 69240780 | 69240893 | Hypomethylated |
| Immune | | | | |
| Liver Resident | chr14 | 61799217 | 61801202 | Hypomethylated |
| Immune | | | | |
| Liver Resident | chr2 | 232396314 | 232396622 | Hypomethylated |
| Immune | | | | |
| Liver Resident | chr13 | 24825770 | 24826000 | Hypomethylated |
| Immune | | | | |
| Liver Resident | chr3 | 134634509 | 134634904 | Hypomethylated |
| Immune | | | | |
| Liver Resident | chr22 | 28501414 | 28501559 | Hypomethylated |
| Immune | | | | |
| Liver Resident | chr8 | 55788511 | 55789245 | Hypomethylated |
| Immune | | | | |
| Liver Resident | chr8 | 129089049 | 129089294 | Hypomethylated |
| Immune | | | | |
| Liver Resident | chr10 | 8373235 | 8373451 | Hypomethylated |
| Immune | | | | |
| Liver Resident | chr11 | 67254108 | 67254405 | Hypomethylated |
| Immune | | | | |
| Liver Resident | chr1 | 228004834 | 228005102 | Hypomethylated |
| Immune | | | | |
| Liver Resident | chr13 | 53507709 | 53507874 | Hypomethylated |
| Immune | | | | |
| Liver Resident | chr3 | 15492693 | 15493298 | Hypomethylated |
| Immune | | | | |
| Liver Resident | chr11 | 119897856 | 119898003 | Hypomethylated |
| Immune | | | | |
| Liver Resident | chr19 | 14231243 | 14232111 | Hypomethylated |
| Immune | | | | |
| Liver Resident | chr8 | 588952 | 589567 | Hypomethylated |
| Immune | | | | |
| Liver Resident | chr12 | 42627503 | 42629139 | Hypomethylated |
| Immune | | | | |
| Liver Resident | chr1 | 182925302 | 182926777 | Hypomethylated |
| Immune | | | | |
| Liver Resident | chr17 | 4079397 | 4079653 | Hypomethylated |
| Immune | | | | |
| Liver Resident | chr20 | 57412376 | 57413281 | Hypomethylated |
| Immune | | | | |
| Liver Resident | chr2 | 11775213 | 11775828 | Hypomethylated |
| Immune | | | | |
| Liver Resident | chr5 | 1793815 | 1794340 | Hypomethylated |
| Immune | | | | |
| Liver Resident | chr6 | 2414426 | 2415245 | Hypomethylated |
| Immune | | | | |
| Liver Resident | chr12 | 9106413 | 9107244 | Hypomethylated |
| Immune | | | | |
| Liver Resident | chr3 | 45984585 | 45986499 | Hypomethylated |
| Immune | | | | |
| Liver Resident | chr3 | 60621448 | 60622527 | Hypomethylated |
| Immune | | | | |
| Liver Resident | chr12 | 133413238 | 133413408 | Hypomethylated |
| Immune | | | | |
| Liver Resident | chr2 | 96933283 | 96933947 | Hypomethylated |
| Immune | | | | |
| Liver Resident | chr5 | 133452036 | 133452371 | Hypomethylated |
| Immune | | | | |
| Liver Resident | chr6 | 158985919 | 158986382 | Hypomethylated |
| Immune | | | | |
| Liver Resident | chr9 | 101754799 | 101755349 | Hypomethylated |
| Immune | | | | |
| Liver Resident | chr10 | 63746813 | 63747406 | Hypomethylated |
| Immune | | | | |
| Liver Resident | chr12 | 122712075 | 122712154 | Hypomethylated |
| Immune | | | | |
| Liver Resident | chr1 | 167486587 | 167487296 | Hypomethylated |
| Immune | | | | |
| Liver Resident | chr7 | 155024838 | 155025349 | Hypomethylated |
| Immune | | | | |
| Liver Resident | chr8 | 10210347 | 10210483 | Hypomethylated |
| Immune | | | | |
| Liver Resident | chr11 | 61672040 | 61672078 | Hypermethylated |
| Immune | | | | |
| Liver Resident | chr2 | 173293597 | 173294219 | Hypermethylated |
| Immune | | | | |
| Liver Resident | chr11 | 64009996 | 64010074 | Hypermethylated |
| Immune | | | | |
| Liver Resident | chr9 | 19050493 | 19050678 | Hypermethylated |
| Immune | | | | |
| Liver Resident | chr10 | 85955136 | 85955226 | Hypermethylated |
| Immune | | | | |
| Lung Epithelial | chr2 | 234394496 | 234394646 | Hypomethylated |
| Lung Epithelial | chr6 | 7204780 | 7204927 | Hypomethylated |
| Lung Epithelial | chr6 | 163731158 | 163731308 | Hypomethylated |
| Lung Epithelial | chr11 | 113216805 | 113217041 | Hypomethylated |
| Lung Epithelial | chr13 | 114304531 | 114304702 | Hypomethylated |
| Lung Epithelial | chr22 | 46929575 | 46929762 | Hypomethylated |
| Lung Epithelial | chr20 | 56296443 | 56296576 | Hypomethylated |
| Lung Epithelial | chr2 | 235237949 | 235238118 | Hypomethylated |
| Lung Epithelial | chr11 | 66454486 | 66454682 | Hypomethylated |
| Lung Epithelial | chr12 | 66983978 | 66984318 | Hypomethylated |
| Lung Epithelial | chr20 | 56296470 | 56296576 | Hypomethylated |
| Lung Epithelial | chr16 | 85517269 | 85517471 | Hypomethylated |
| Lung Epithelial | chr17 | 79953059 | 79953137 | Hypomethylated |
| Lung Epithelial | chr1 | 2266262 | 2266414 | Hypomethylated |
| Lung Epithelial | chr12 | 26261448 | 26262385 | Hypomethylated |
| Lung Epithelial | chr14 | 104048108 | 104048201 | Hypomethylated |
| Lung Epithelial | chr13 | 111935187 | 111935468 | Hypomethylated |
| Lung Epithelial | chr7 | 2770561 | 2770802 | Hypomethylated |
| Lung Epithelial | chr9 | 136728413 | 136728520 | Hypomethylated |
| Lung Epithelial | chr10 | 1257678 | 1257979 | Hypomethylated |
| Lung Epithelial | chr10 | 112889204 | 112889350 | Hypomethylated |
| Lung Epithelial | chr17 | 80247859 | 80248026 | Hypomethylated |
| Lung Epithelial | chr11 | 111171223 | 111172653 | Hypomethylated |
| Lung Epithelial | chr6 | 46889597 | 46889761 | Hypomethylated |
| Lung Epithelial | chr8 | 142157078 | 142157174 | Hypomethylated |
| Lung Epithelial | chr17 | 9088165 | 9088467 | Hypomethylated |
| Lung Epithelial | chr2 | 234393573 | 234394479 | Hypomethylated |
| Lung Epithelial | chr16 | 677895 | 678174 | Hypomethylated |
| Lung Epithelial | chr17 | 71548667 | 71548755 | Hypomethylated |
| Lung Epithelial | chr1 | 2059898 | 2060168 | Hypomethylated |
| Lung Epithelial | chr20 | 56296660 | 56296827 | Hypomethylated |
| Lung Epithelial | chr2 | 234394372 | 234394479 | Hypomethylated |
| Lung Epithelial | chr16 | 12354976 | 12355424 | Hypomethylated |
| Lung Epithelial | chr7 | 33725751 | 33726087 | Hypomethylated |
| Lung Epithelial | chr10 | 13771352 | 13771658 | Hypomethylated |
| Lung Epithelial | chr22 | 46838804 | 46839245 | Hypomethylated |
| Lung Epithelial | chr12 | 94093563 | 94093982 | Hypomethylated |
| Lung Epithelial | chr6 | 155568960 | 155569279 | Hypomethylated |
| Lung Epithelial | chr13 | 114115128 | 114115433 | Hypomethylated |
| Lung Epithelial | chr4 | 1977200 | 1977236 | Hypomethylated |
| Lung Epithelial | chr4 | 8248193 | 8248391 | Hypomethylated |
| Lung Epithelial | chr6 | 46743464 | 46744227 | Hypomethylated |
| Lung Epithelial | chr13 | 98869837 | 98870145 | Hypomethylated |
| Lung Epithelial | chr16 | 3763054 | 3763348 | Hypomethylated |
| Lung Epithelial | chr19 | 8554999 | 8555062 | Hypomethylated |
| Lung Epithelial | chr7 | 5262422 | 5262575 | Hypomethylated |
| Lung Epithelial | chr17 | 41024622 | 41025029 | Hypomethylated |
| Lung Epithelial | chr21 | 43546441 | 43546655 | Hypomethylated |
| Lung Epithelial | chr4 | 8436008 | 8436223 | Hypomethylated |
| Lung Epithelial | chr22 | 19754439 | 19754839 | Hypermethylated |
| Megakaryocyte | chr16 | 1814980 | 1815151 | Hypomethylated |
| Megakaryocyte | chr16 | 3188474 | 3188511 | Hypomethylated |
| Megakaryocyte | chr1 | 7911113 | 7911210 | Hypomethylated |
| Megakaryocyte | chr10 | 467813 | 468083 | Hypomethylated |
| Megakaryocyte | chr19 | 1872715 | 1873024 | Hypomethylated |
| Megakaryocyte | chr9 | 71681914 | 71682266 | Hypomethylated |
| Megakaryocyte | chr14 | 103566265 | 103566681 | Hypomethylated |
| Megakaryocyte | chr16 | 88568536 | 88568989 | Hypomethylated |
| Megakaryocyte | chr17 | 61498395 | 61498815 | Hypomethylated |
| Megakaryocyte | chr8 | 145674184 | 145674298 | Hypomethylated |
| Megakaryocyte | chr12 | 8941393 | 8941494 | Hypomethylated |
| Megakaryocyte | chr5 | 481723 | 481794 | Hypomethylated |
| Megakaryocyte | chr2 | 239023319 | 239023500 | Hypomethylated |
| Megakaryocyte | chr5 | 37208967 | 37209202 | Hypomethylated |
| Megakaryocyte | chr11 | 68522449 | 68522793 | Hypomethylated |
| Megakaryocyte | chr19 | 56113869 | 56114292 | Hypomethylated |
| Megakaryocyte | chr8 | 142180962 | 142181506 | Hypomethylated |
| Megakaryocyte | chr14 | 92956120 | 92956450 | Hypomethylated |
| Megakaryocyte | chr14 | 105167146 | 105167290 | Hypomethylated |
| Megakaryocyte | chr19 | 16629936 | 16630066 | Hypomethylated |
| Megakaryocyte | chr19 | 14066489 | 14067377 | Hypomethylated |
| Megakaryocyte | chr5 | 37209212 | 37209381 | Hypomethylated |
| Megakaryocyte | chr7 | 1785741 | 1786129 | Hypomethylated |
| Megakaryocyte | chr7 | 137230659 | 137230997 | Hypomethylated |
| Megakaryocyte | chr8 | 10273760 | 10274076 | Hypomethylated |
| Megakaryocyte | chr7 | 631951 | 632605 | Hypomethylated |
| Megakaryocyte | chr7 | 632621 | 632817 | Hypomethylated |
| Megakaryocyte | chr11 | 63977307 | 63977540 | Hypomethylated |
| Megakaryocyte | chr1 | 1871823 | 1871974 | Hypomethylated |
| Megakaryocyte | chr19 | 5593710 | 5594329 | Hypomethylated |
| Megakaryocyte | chr22 | 39909921 | 39910015 | Hypomethylated |
| Megakaryocyte | chr11 | 68898452 | 68898667 | Hypomethylated |
| Megakaryocyte | chr19 | 12799867 | 12800212 | Hypomethylated |
| Megakaryocyte | chr19 | 49254245 | 49254406 | Hypomethylated |
| Megakaryocyte | chr7 | 149129744 | 149130209 | Hypomethylated |
| Megakaryocyte | chr15 | 74714681 | 74715017 | Hypomethylated |
| Megakaryocyte | chr19 | 50376109 | 50376389 | Hypomethylated |
| Megakaryocyte | chr8 | 145814448 | 145814812 | Hypomethylated |
| Megakaryocyte | chr14 | 102963357 | 102963929 | Hypomethylated |
| Megakaryocyte | chr16 | 88557690 | 88558056 | Hypomethylated |
| Megakaryocyte | chr2 | 179640101 | 179641010 | Hypomethylated |
| Megakaryocyte | chr3 | 194968078 | 194968601 | Hypomethylated |
| Megakaryocyte | chr5 | 1924041 | 1924100 | Hypomethylated |
| Megakaryocyte | chr5 | 53223677 | 53224181 | Hypomethylated |
| Megakaryocyte | chr8 | 141312889 | 141313313 | Hypomethylated |
| Megakaryocyte | chr16 | 1545317 | 1545676 | Hypomethylated |
| Megakaryocyte | chr3 | 194835984 | 194836405 | Hypomethylated |
| Megakaryocyte | chr8 | 53323520 | 53323719 | Hypomethylated |
| Megakaryocyte | chr16 | 85551464 | 85551806 | Hypomethylated |
| Megakaryocyte | chr16 | 86755593 | 86756024 | Hypomethylated |
| Monocytes and | chr3 | 196351807 | 196352171 | Hypomethylated |
| Macrophage | | | | |
| Monocytes and | chr12 | 6659484 | 6659682 | Hypomethylated |
| Macrophage | | | | |
| Monocytes and | chr13 | 32888845 | 32889052 | Hypomethylated |
| Macrophage | | | | |
| Monocytes and | chr3 | 195897812 | 195898123 | Hypomethylated |
| Macrophage | | | | |
| Monocytes and | chr19 | 8568502 | 8568649 | Hypomethylated |
| Macrophage | | | | |
| Monocytes and | chr16 | 85577692 | 85577882 | Hypomethylated |
| Macrophage | | | | |
| Monocytes and | chr12 | 132469818 | 132470033 | Hypomethylated |
| Macrophage | | | | |
| Monocytes and | chr17 | 80581522 | 80581992 | Hypomethylated |
| Macrophage | | | | |
| Monocytes and | chr3 | 128370161 | 128370565 | Hypomethylated |
| Macrophage | | | | |
| Monocytes and | chr4 | 3531428 | 3531642 | Hypomethylated |
| Macrophage | | | | |
| Monocytes and | chr9 | 95799785 | 95800025 | Hypomethylated |
| Macrophage | | | | |
| Monocytes and | chr18 | 74824341 | 74824414 | Hypomethylated |
| Macrophage | | | | |
| Monocytes and | chr12 | 56731566 | 56731735 | Hypomethylated |
| Macrophage | | | | |
| Monocytes and | chr16 | 56892384 | 56892556 | Hypomethylated |
| Macrophage | | | | |
| Monocytes and | chr16 | 85873312 | 85873440 | Hypomethylated |
| Macrophage | | | | |
| Monocytes and | chr2 | 134866101 | 134866288 | Hypomethylated |
| Macrophage | | | | |
| Monocytes and | chr8 | 602054 | 602159 | Hypomethylated |
| Macrophage | | | | |
| Monocytes and | chr5 | 79422700 | 79423348 | Hypomethylated |
| Macrophage | | | | |
| Monocytes and | chr16 | 3597075 | 3597441 | Hypomethylated |
| Macrophage | | | | |
| Monocytes and | chr6 | 26385502 | 26385842 | Hypomethylated |
| Macrophage | | | | |
| Monocytes and | chr14 | 69091384 | 69091490 | Hypomethylated |
| Macrophage | | | | |
| Monocytes and | chr2 | 240225004 | 240225176 | Hypomethylated |
| Macrophage | | | | |
| Monocytes and | chr21 | 45773615 | 45773734 | Hypomethylated |
| Macrophage | | | | |
| Monocytes and | chr11 | 47350123 | 47350267 | Hypomethylated |
| Macrophage | | | | |
| Monocytes and | chr19 | 2073026 | 2073176 | Hypomethylated |
| Macrophage | | | | |
| Monocytes and | chr22 | 37309307 | 37309488 | Hypomethylated |
| Macrophage | | | | |
| Monocytes and | chr2 | 28697740 | 28697866 | Hypomethylated |
| Macrophage | | | | |
| Neuron | chr6 | 163558238 | 163558675 | Hypomethylated |
| Neuron | chr1 | 2006859 | 2007311 | Hypomethylated |
| Neuron | chr16 | 81731208 | 81731484 | Hypomethylated |
| Neuron | chr9 | 138775499 | 138775772 | Hypomethylated |
| Neuron | chr17 | 30815173 | 30815470 | Hypomethylated |
| Neuron | chr8 | 143463390 | 143463895 | Hypomethylated |
| Neuron | chr1 | 14113094 | 14113343 | Hypomethylated |
| Neuron | chr1 | 110082688 | 110083518 | Hypomethylated |
| Neuron | chr17 | 76675816 | 76676403 | Hypomethylated |
| Neuron | chr18 | 42324837 | 42325188 | Hypomethylated |
| Neuron | chr3 | 47932918 | 47934038 | Hypomethylated |
| Neuron | chr11 | 117232011 | 117232455 | Hypomethylated |
| Neuron | chr17 | 76818594 | 76819359 | Hypomethylated |
| Neuron | chr10 | 131694516 | 131694773 | Hypomethylated |
| Neuron | chr7 | 636539 | 636950 | Hypomethylated |
| Neuron | chr11 | 6425899 | 6426501 | Hypomethylated |
| Neuron | chr1 | 2236562 | 2236748 | Hypomethylated |
| Neuron | chr1 | 6363384 | 6363883 | Hypomethylated |
| Neuron | chr1 | 111145683 | 111147040 | Hypomethylated |
| Neuron | chr2 | 1828347 | 1828664 | Hypomethylated |
| Neuron | chr9 | 138627849 | 138628084 | Hypomethylated |
| Neuron | chr10 | 3283414 | 3283766 | Hypomethylated |
| Neuron | chr1 | 2238334 | 2238847 | Hypomethylated |
| Neuron | chr19 | 4770747 | 4770983 | Hypomethylated |
| Neuron | chr7 | 150816696 | 150817291 | Hypomethylated |
| Neuron | chr17 | 77083111 | 77083194 | Hypomethylated |
| Neuron | chr2 | 43019573 | 43020395 | Hypermethylated |
| Neuron | chr3 | 49941216 | 49941579 | Hypermethylated |
| Neuron | chr19 | 1467071 | 1467140 | Hypermethylated |
| Neuron | chr6 | 166267918 | 166268069 | Hypermethylated |
| Neuron | chr11 | 31841762 | 31842091 | Hypermethylated |
| Neuron | chr17 | 8924080 | 8924226 | Hypermethylated |
| Neuron | chr21 | 34755391 | 34755801 | Hypermethylated |
| Neuron | chr19 | 38886319 | 38886635 | Hypermethylated |
| Neuron | chr19 | 46142860 | 46142980 | Hypermethylated |
| Neuron | chr9 | 38672385 | 38672729 | Hypermethylated |
| Neuron | chr10 | 103534498 | 103534585 | Hypermethylated |
| Neuron | chr11 | 64066758 | 64067406 | Hypermethylated |
| Neuron | chr12 | 130529868 | 130530228 | Hypermethylated |
| Neuron | chr19 | 1455008 | 1456059 | Hypermethylated |
| Neuron | chr17 | 72352876 | 72353282 | Hypermethylated |
| Neuron | chr19 | 46379859 | 46380198 | Hypermethylated |
| Neuron | chr4 | 8200669 | 8201295 | Hypermethylated |
| Neuron | chr12 | 49366026 | 49366367 | Hypermethylated |
| Neuron | chr7 | 140346986 | 140347127 | Hypermethylated |
| Neuron | chr7 | 131514718 | 131515081 | Hypermethylated |
| Neuron | chr11 | 62693619 | 62693987 | Hypermethylated |
| Neuron | chr8 | 145697840 | 145698117 | Hypermethylated |
| Neuron | chr2 | 97151733 | 97152183 | Hypermethylated |
| Neuron | chr14 | 70038530 | 70038749 | Hypermethylated |
| Natural Killer | chr6 | 159457324 | 159457551 | Hypomethylated |
| Natural Killer | chr6 | 10733158 | 10733571 | Hypomethylated |
| Natural Killer | chr17 | 1104805 | 1105072 | Hypomethylated |
| Natural Killer | chr5 | 10377133 | 10377320 | Hypomethylated |
| Natural Killer | chr2 | 426239 | 426502 | Hypomethylated |
| Natural Killer | chr6 | 158985966 | 158986382 | Hypomethylated |
| Natural Killer | chr16 | 2892965 | 2893055 | Hypomethylated |
| Natural Killer | chr10 | 72358669 | 72358804 | Hypomethylated |
| Natural Killer | chr10 | 72362893 | 72363273 | Hypomethylated |
| Natural Killer | chr19 | 4657680 | 4658152 | Hypomethylated |
| Natural Killer | chr13 | 114874439 | 114874735 | Hypomethylated |
| Natural Killer | chr3 | 39322103 | 39322776 | Hypomethylated |
| Natural Killer | chr21 | 47845235 | 47846073 | Hypomethylated |
| Natural Killer | chr22 | 46685653 | 46686131 | Hypomethylated |
| Natural Killer | chr1 | 11395635 | 11395863 | Hypomethylated |
| Natural Killer | chr11 | 120827449 | 120827536 | Hypomethylated |
| Natural Killer | chr19 | 10226728 | 10226846 | Hypomethylated |
| Natural Killer | chr13 | 77565285 | 77565577 | Hypomethylated |
| Natural Killer | chr10 | 72357918 | 72358611 | Hypomethylated |
| Natural Killer | chr16 | 89336192 | 89336628 | Hypomethylated |
| Natural Killer | chr21 | 47830003 | 47830198 | Hypomethylated |
| Natural Killer | chr16 | 84553497 | 84553694 | Hypomethylated |
| Pancreatic | chr7 | 97841826 | 97842223 | Hypomethylated |
| Pancreatic | chr17 | 80395185 | 80395451 | Hypomethylated |
| Pancreatic | chr7 | 97843854 | 97844802 | Hypomethylated |
| Pancreatic | chr16 | 3706036 | 3706908 | Hypomethylated |
| Pancreatic | chr1 | 22303212 | 22303543 | Hypomethylated |
| Pancreatic | chr9 | 139394558 | 139395025 | Hypomethylated |
| Pancreatic | chr19 | 56658298 | 56658723 | Hypomethylated |
| Pancreatic | chr6 | 35762792 | 35763566 | Hypomethylated |
| Pancreatic | chr11 | 794340 | 794623 | Hypomethylated |
| Pancreatic | chr4 | 186742139 | 186742364 | Hypomethylated |
| Pancreatic | chr16 | 75252683 | 75252951 | Hypomethylated |
| Pancreatic | chr17 | 78337515 | 78338076 | Hypomethylated |
| Pancreatic | chr9 | 135929763 | 135929928 | Hypomethylated |
| Pancreatic | chr9 | 135944497 | 135946085 | Hypomethylated |
| Pancreatic | chr8 | 145685382 | 145685798 | Hypomethylated |
| Pancreatic | chr16 | 630101 | 630351 | Hypomethylated |
| Pancreatic | chr17 | 77923890 | 77924179 | Hypomethylated |
| Pancreatic | chr8 | 103942869 | 103943309 | Hypomethylated |
| Pancreatic | chr17 | 705656 | 706287 | Hypomethylated |
| Pancreatic | chr19 | 39691205 | 39691581 | Hypomethylated |
| Pancreatic | chr19 | 49253497 | 49253771 | Hypomethylated |
| Pancreatic | chr9 | 139393948 | 139394113 | Hypomethylated |
| Pancreatic | chr16 | 75255120 | 75255591 | Hypomethylated |
| Pancreatic | chr10 | 130546138 | 130546266 | Hypomethylated |
| Pancreatic | chr16 | 88473427 | 88473645 | Hypomethylated |
| Pancreatic | chr5 | 1702312 | 1702471 | Hypomethylated |
| Pancreatic | chr5 | 10757768 | 10758645 | Hypomethylated |
| Pancreatic | chr7 | 97846514 | 97847025 | Hypomethylated |
| Pancreatic | chr11 | 1321822 | 1322132 | Hypomethylated |
| Pancreatic | chr12 | 110353054 | 110353218 | Hypomethylated |
| Pancreatic | chr12 | 126676605 | 126676791 | Hypomethylated |
| Pancreatic | chr17 | 37328203 | 37328335 | Hypomethylated |
| Pancreatic | chr19 | 49254245 | 49254406 | Hypomethylated |
| Pancreatic | chr7 | 97857387 | 97857615 | Hypomethylated |
| Pancreatic | chr1 | 22854310 | 22854475 | Hypomethylated |
| Pancreatic | chr7 | 150034206 | 150034367 | Hypomethylated |
| Pancreatic | chr1 | 24463665 | 24463848 | Hypomethylated |
| Pancreatic | chr6 | 35764704 | 35765094 | Hypomethylated |
| Pancreatic | chr20 | 17633665 | 17633859 | Hypomethylated |
| Pancreatic | chr4 | 1372943 | 1373417 | Hypomethylated |
| Pancreatic | chr7 | 156797845 | 156798469 | Hypermethylated |
| Pancreatic | chr13 | 28496765 | 28497184 | Hypermethylated |
| Pancreatic | chr13 | 28497826 | 28498280 | Hypermethylated |
| Pancreatic | chr11 | 119227020 | 119227708 | Hypermethylated |
| Pancreatic | chr14 | 105714520 | 105715440 | Hypermethylated |
| Pancreatic | chr7 | 157476754 | 157477033 | Hypermethylated |
| Pancreatic | chr15 | 53089968 | 53090394 | Hypermethylated |
| Pancreatic | chr19 | 47960704 | 47960978 | Hypermethylated |
| Pancreatic | chr12 | 65218052 | 65218948 | Hypermethylated |
| Pancreatic | chr13 | 28491265 | 28491526 | Hypermethylated |
| Prostate Epithelial | chr20 | 18295551 | 18295861 | Hypomethylated |
| Prostate Epithelial | chr7 | 2054500 | 2055180 | Hypomethylated |
| Prostate Epithelial | chr17 | 40823869 | 40824215 | Hypomethylated |
| Prostate Epithelial | chr17 | 9088165 | 9088467 | Hypomethylated |
| Prostate Epithelial | chr22 | 40415244 | 40415401 | Hypomethylated |
| Prostate Epithelial | chr1 | 27220656 | 27221119 | Hypomethylated |
| Prostate Epithelial | chr7 | 5549534 | 5549731 | Hypomethylated |
| Prostate Epithelial | chr16 | 81533203 | 81533549 | Hypomethylated |
| Prostate Epithelial | chr11 | 70022863 | 70023099 | Hypomethylated |
| Prostate Epithelial | chr7 | 1491787 | 1491952 | Hypomethylated |
| Prostate Epithelial | chr8 | 143386225 | 143386548 | Hypomethylated |
| Prostate Epithelial | chr8 | 142235391 | 142235926 | Hypomethylated |
| Prostate Epithelial | chr19 | 8554999 | 8555062 | Hypomethylated |
| Prostate Epithelial | chr10 | 116080333 | 116080495 | Hypomethylated |
| Prostate Epithelial | chr19 | 14636919 | 14637391 | Hypomethylated |
| Prostate Epithelial | chr9 | 140708598 | 140709066 | Hypomethylated |
| Prostate Epithelial | chr16 | 23158676 | 23159171 | Hypomethylated |
| Prostate Epithelial | chr19 | 30298144 | 30298342 | Hypomethylated |
| Prostate Epithelial | chr6 | 6894045 | 6894183 | Hypomethylated |
| Prostate Epithelial | chr18 | 77231461 | 77231655 | Hypomethylated |
| Prostate Epithelial | chr20 | 60953594 | 60953740 | Hypomethylated |
| Prostate Epithelial | chr1 | 17307773 | 17308093 | Hypomethylated |
| Prostate Epithelial | chr1 | 19208516 | 19208778 | Hypomethylated |
| Prostate Epithelial | chr2 | 174147832 | 174148599 | Hypomethylated |
| Prostate Epithelial | chr8 | 8748227 | 8749041 | Hypomethylated |
| Prostate Epithelial | chr11 | 129802069 | 129803188 | Hypomethylated |
| Prostate Epithelial | chr3 | 124859057 | 124859348 | Hypomethylated |
| Prostate Epithelial | chr3 | 197121225 | 197121420 | Hypomethylated |
| Prostate Epithelial | chr19 | 48796188 | 48796381 | Hypomethylated |
| Prostate Epithelial | chr4 | 1225408 | 1225653 | Hypomethylated |
| Prostate Epithelial | chr19 | 1425504 | 1425707 | Hypomethylated |
| Prostate Epithelial | chr1 | 3615514 | 3615746 | Hypomethylated |
| Prostate Epithelial | chr10 | 5567123 | 5567503 | Hypomethylated |
| Prostate Epithelial | chr10 | 81915434 | 81915647 | Hypomethylated |
| Prostate Epithelial | chr11 | 120007968 | 120008155 | Hypomethylated |
| Prostate Epithelial | chr20 | 18167947 | 18168360 | Hypomethylated |
| Prostate Epithelial | chr7 | 551344 | 551759 | Hypomethylated |
| Prostate Epithelial | chr1 | 38493014 | 38493557 | Hypomethylated |
| Prostate Epithelial | chr6 | 33740395 | 33740572 | Hypomethylated |
| Prostate Epithelial | chr7 | 26480584 | 26480922 | Hypomethylated |
| Prostate Epithelial | chr10 | 133796508 | 133796631 | Hypomethylated |
| Prostate Epithelial | chr22 | 43463194 | 43463331 | Hypomethylated |
| Prostate Epithelial | chr22 | 47023029 | 47023196 | Hypomethylated |
| Prostate Epithelial | chr1 | 2359816 | 2359937 | Hypomethylated |
| Prostate Epithelial | chr21 | 46232702 | 46232873 | Hypomethylated |
| Prostate Epithelial | chr22 | 43164347 | 43164751 | Hypomethylated |
| Prostate Epithelial | chr4 | 1317409 | 1317679 | Hypomethylated |
| Prostate Epithelial | chr10 | 30317521 | 30317689 | Hypomethylated |
| Prostate Epithelial | chr16 | 73099735 | 73099893 | Hypermethylated |
| Prostate Epithelial | chr6 | 10420567 | 10421060 | Hypermethylated |
| Skeletal Muscular | chr7 | 148497704 | 148497985 | Hypomethylated |
| Skeletal Muscular | chr18 | 46289370 | 46289545 | Hypomethylated |
| Skeletal Muscular | chr10 | 99330013 | 99330204 | Hypomethylated |
| Skeletal Muscular | chr11 | 129613860 | 129614016 | Hypomethylated |
| Skeletal Muscular | chr1 | 9324223 | 9324364 | Hypomethylated |
| Skeletal Muscular | chr18 | 55922618 | 55922960 | Hypomethylated |
| Skeletal Muscular | chr3 | 14582440 | 14582904 | Hypomethylated |
| Skeletal Muscular | chr17 | 78724929 | 78725376 | Hypomethylated |
| Skeletal Muscular | chr17 | 3769455 | 3770035 | Hypomethylated |
| Skeletal Muscular | chr10 | 23353063 | 23354028 | Hypomethylated |
| Skeletal Muscular | chr18 | 55923895 | 55924181 | Hypomethylated |
| Skeletal Muscular | chr1 | 1490850 | 1491199 | Hypomethylated |
| Skeletal Muscular | chr2 | 201342380 | 201342659 | Hypomethylated |
| Skeletal Muscular | chr7 | 611233 | 612221 | Hypomethylated |
| Skeletal Muscular | chr3 | 52869808 | 52871468 | Hypomethylated |
| Skeletal Muscular | chr17 | 65040730 | 65041120 | Hypomethylated |
| Skeletal Muscular | chr7 | 1351324 | 1351753 | Hypomethylated |
| Skeletal Muscular | chr17 | 76672617 | 76673183 | Hypomethylated |
| Skeletal Muscular | chr1 | 54856305 | 54856608 | Hypomethylated |
| Skeletal Muscular | chr4 | 25097386 | 25097706 | Hypomethylated |
| Skeletal Muscular | chr4 | 100572972 | 100573578 | Hypomethylated |
| Skeletal Muscular | chr7 | 613015 | 613422 | Hypomethylated |
| Skeletal Muscular | chr14 | 20903959 | 20904321 | Hypomethylated |
| Skeletal Muscular | chr17 | 79898313 | 79898988 | Hypomethylated |
| Skeletal Muscular | chr5 | 138730990 | 138731670 | Hypomethylated |
| Skeletal Muscular | chr17 | 11461316 | 11461624 | Hypomethylated |
| Skeletal Muscular | chr7 | 1351335 | 1351753 | Hypomethylated |
| Skeletal Muscular | chr7 | 56149083 | 56150012 | Hypomethylated |
| Skeletal Muscular | chr14 | 104636600 | 104636829 | Hypomethylated |
| Skeletal Muscular | chr7 | 612876 | 613698 | Hypomethylated |
| Skeletal Muscular | chr10 | 134894695 | 134894939 | Hypomethylated |
| Skeletal Muscular | chr17 | 11461335 | 11461638 | Hypomethylated |
| Skeletal Muscular | chr4 | 126572121 | 126572399 | Hypomethylated |
| Skeletal Muscular | chr16 | 89258456 | 89259792 | Hypermethylated |
| Mature T | chr13 | 24825869 | 24826000 | Hypomethylated |
| Mature T | chr22 | 37544974 | 37545667 | Hypomethylated |
| Mature T | chr1 | 17054071 | 17054128 | Hypomethylated |
| Mature T | chr10 | 13330036 | 13330429 | Hypomethylated |
| Mature T | chr10 | 1156420 | 1156511 | Hypomethylated |
| Mature T | chr2 | 85667088 | 85667542 | Hypomethylated |
| Mature T | chr13 | 111841565 | 111842079 | Hypomethylated |
| Mature T | chr14 | 99715693 | 99716236 | Hypomethylated |
| Mature T | chr7 | 625073 | 625320 | Hypomethylated |
| Mature T | chr11 | 60775001 | 60775404 | Hypermethylated |
| Mature T | chr3 | 42113514 | 42114020 | Hypermethylated |

*The start and end points of the genomic region is with reference to the Homo sapiens full genome as provided by University of California Santa Cruz, version hg19 (Genome Reference Consortium GRCh37, February 2009).

### Determination of Source Origin

Determination of the source origin of the cfDNA comprises genotyping the cfDNA in order to obtain a genotype profile of the cfDNA. The genotype profile of the cfDNA can be compared with the genotype profile of the donor and/or the genotype profile of the subject to determine whether the cfDNA is originating from foreign biological material from the donor or from the subject.

In some embodiments, genotyping comprises detection, quantitation, or both detection and quantitation, of polymorphic markers. Examples of polymorphic markers include, but are not limited to, SNPs, restriction fragment length polymorphisms (RFLPs), variable number of tandem repeats (VNTRs), short tandem repeats (STRs), hypervariable regions, minisatellites, dinucleotide repeats, trinucleotide repeats, tetranucleotide repeats, simple sequence repeats, and insertion elements such as Alu. In preferred embodiments, genotyping comprises detection of SNPs.

Thus, in some embodiments, the genotype profile comprises a polymorphic marker profile. In certain embodiments, the genotype profile comprises a SNP profile. For instance, the SNP profile comprises at least the universal SNP positions determined by the 1000 Genomes Project, HapMap, or both (see, e.g., Huang et al., 2018).

Examples of methods that can be used in genotyping include, but are not limited to, whole genome sequencing. sequencing of a sufficient number of regions of the genome, and polymorphisms arrays (e.g., SNP arrays). In some embodiments, genotyping the donor and/or the subject may comprise sequencing at least about 100, or at least about 125, or at least about 150, or at least about 175, or at least about 200, or at least about 225, or at least about 250, or at least about 275, or at least about 300 regions of the genome, for instance, by amplicon sequencing or by hybridization capture sequencing. Other methods include, but are not limited to, polymerase chain reaction (PCR) techniques (e.g., quantitative PCR, quantitative fluorescent PCR, multiplex fluorescent PCR, real time PCR, single cell PCR, restriction fragment length polymorphism PCR, etc.), and the use of arrays (e.g., SNPs arrays).

In some embodiments, the methods of the present invention further comprises genotyping the subject to obtain a genotype profile of the subject. In some embodiments, the methods of the present invention further comprises genotyping the donor to obtain a genotype profile of the donor.

Comparison of the genotype profile of the cfDNA with the genotype profile of the donor and/or the genotype profile of the subject may comprise identifying the presence of the same polymorphic markers (e.g., same SNPs) in the cfDNA, or a portion thereof, and in the genotype profile of the donor and/or the genotype profile of the subject. In some embodiments, the portion of the cfDNA is the same portion of cfDNA in which methylation patterns identifying the cellular origin is determined. In some embodiments, the polymorphic profile is obtained for the same one or more portions of the sequence of the cell-free DNA of which methylation patterns were identified. Thus, cell death is detected when the one or more portions of cell-free DNA has both a cellular origin of the type of foreign biological material that was received from the donor, and a source origin of the donor.

### EXAMPLES

### Example 1

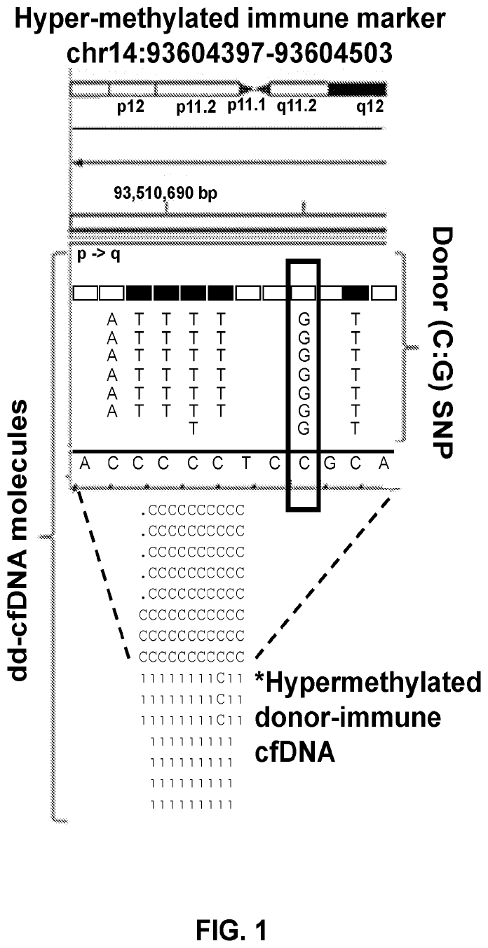

Donor-SNPs identified from donor and host liver biopsies were used to detect dd-cfDNA molecules in the circulation of three patients. The dd-cfDNA levels were found to correlate with alanine transaminase/aspartate transaminase levels as well as with predicted hepatocyte-derived molecules based on cfDNA methylation. Overlay of dd-cfDNA molecules with immune cell methylation markers allowed for detection of both host and donor-immune cell-death in peripheral blood. As shown in FIG. 1. a donor-specific C:G SNP was found based on WGS analysis of donor and host liver tissues. CfDNA molecules with the G nucleotide at this position were extracted and intersected with immune cell-specific hyper-methylated marker at chr14:93604397-93604503. 63% of cfDNA molecules with immune cell methylation pattern, contained the donor-SNP (FIG. 1).

This data shows cfDNAs from small cell subpopulations can be detected in the donor liver. Also, the data shows that it is possible to detect and quantitate donor immune cell cfDNA that carries immune DNA methylation patterns overlapping with donor-SNPs. The study provides an example of integration of genetic and epigenetic markers to identify donor cell death from tissue resident immune cells.

### Example 2

A study was conducted in patients undergoing a liver transplant (n=24; nine of the patients had FC, or biopsy-verified, graft dysfunction). Blood was drawn pre-operation on Day 0, post-operation on Day 0, and on Days 7 and 30.

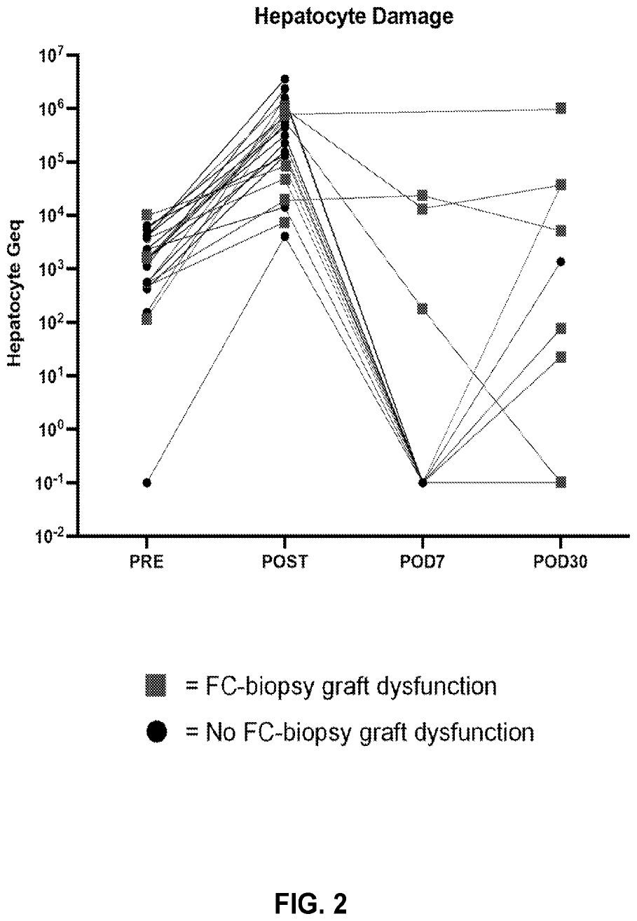

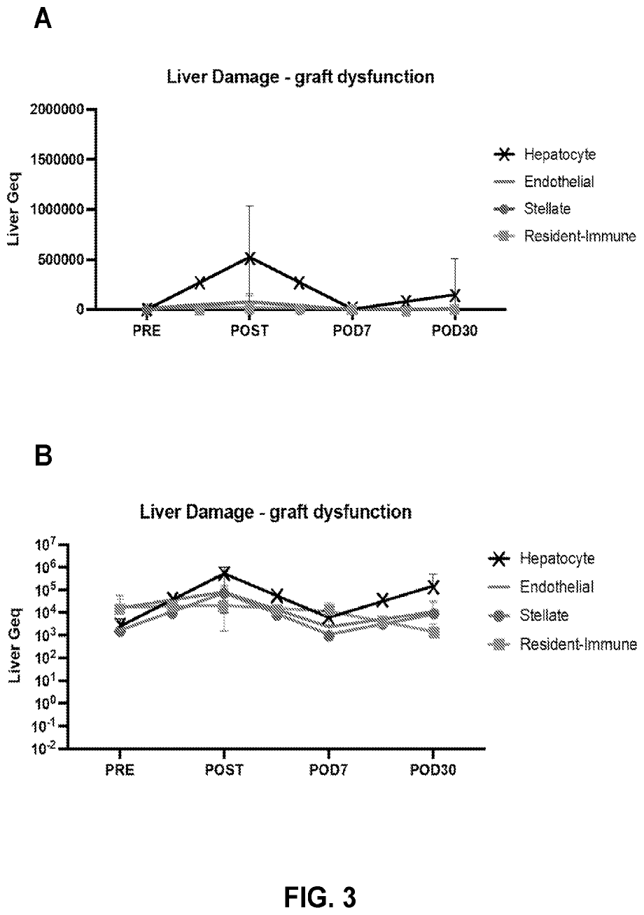

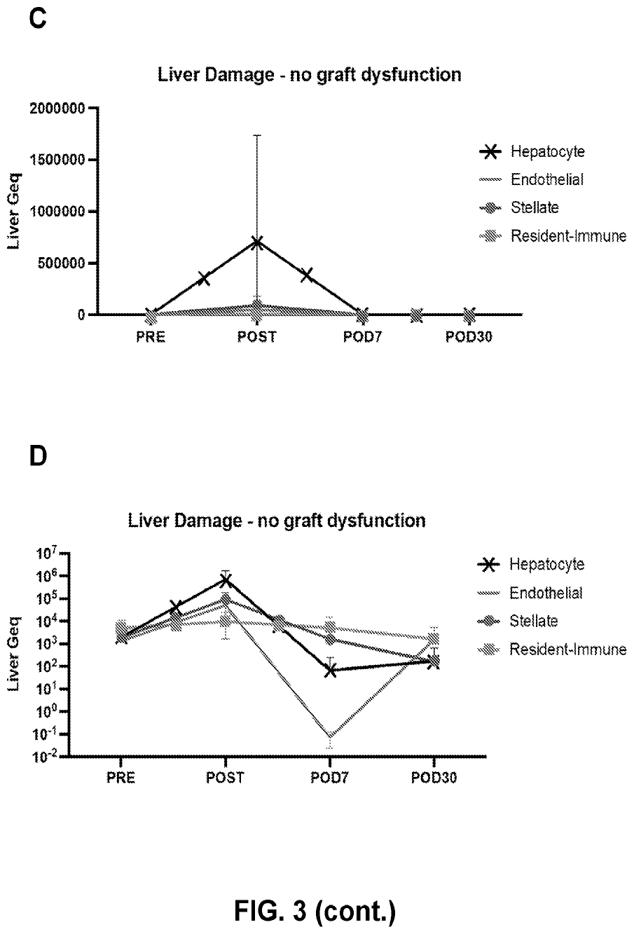

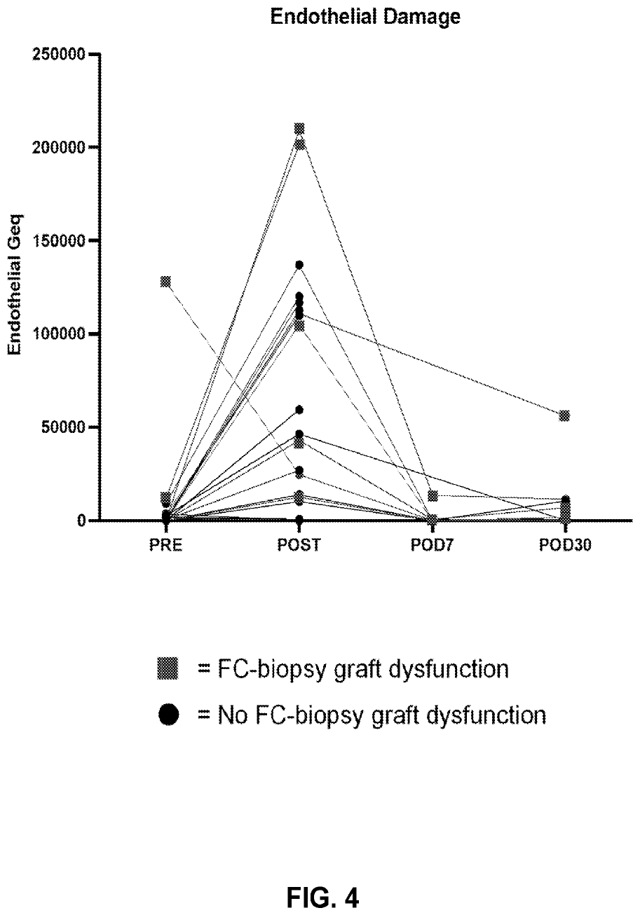

For liver cell-type specific patterns, cfDNA molecules were identified with methylation patterns to indicate they originated from those donor cells. For example, FIG. 2 shows elevated levels of hepatocyte cfDNA after the operation, indicating tissue damage. FIG. 3 shows liver damage as indicated by different liver cell types both with and without graft dysfunction. FIG. 4 shows that endothelial cell damage also occurred post-operation.

These results demonstrate the use of methylation pattern analysis of cfDNA to assess cell damage of transplanted tissue.

### REFERENCES

Bergman Y and Cedar H. DNA methylation dynamics in health and disease. Nature Structural & Molecular Biology 20: 274-281 (2013).

Bock C. Analysing and interpreting DNA methylation data. Nature Reviews Genetics 13:705-719 (2012).

Crowley E, et al. Liquid biopsy: monitoring cancer-genetics in the blood. Nature Reviews Clinical Oncology 10: 472-484 (2013).

Dietrich D. DNA methylation analysis from body fluids. Methods in Molecular Biology 1655: 239-249 (2018).

Fernandez AF, et al. A DNA methylation fingerprint of 1628 human samples. Genome Research 22: 407-419 (2012).

Guo S, et al. Identification of methylation haplotype blocks aids in deconvolution of heterogeneous tissue samples and tumor tissue-of-origin mapping from plasma DNA. Nature Genetics 49: 635-642 (2017).

Huang E, et al. Genome-wide screen for universal individual identification SNPs based on the HapMap and 1000 Genomes databases. Scientific Reports 8: 5553 (2018).

Lehmann-Werman R, et al. Identification of tissue-specific cell death using methylation patterns of circulating DNA. Proceedings of the National Academy of Sciences of the United States of America 113: E1826-E1834 (2016).

Lo YMD, et al. Quantitative analysis of fetal DNA in maternal plasma and serum: implications for noninvasive prenatal diagnosis. American Journal of Human Genetics 62: 768-775 (1998).

Moran S, et al. Precision medicine based on epigenomics: the paradigm of carcinoma of unknown primary. Nature Reviews Clinical Oncology 14: 682-694 (2017).

Park J-L, et al. Identification of body fluid-specific DNA methylation markers for use in forensic science. Forensic Science International: Genetics 13: 147-153 (2014).

Plongthongkum N, et al. Advances in the profiling of DNA modifications: cytosine methylation and beyond. Nature Reviews Genetics 15: 647-661 (2014).

Shiels PG, et al. The role of epigenetics in renal ageing. Nature Reviews Nephrology 13: 471-482 (2017).

Si X, et al. DNA methylation as a potential diagnosis indicator for rapid discrimination of rare cancer cells and normal cells. Scientific Reports 5: 11882 (2015).

Sun K, et al. Plasma DNA tissue mapping by genome-wide methylation sequencing for noninvasive prenatal, cancer, and transplantation assessments. Proceedings of the National Academy of Sciences of the United States of America 112: E5503-5512 2015).

Wilmot B, et al. Methylomic analysis of salivary DNA in childhood ADHD identifies altered DNA methylation in VIPR2. Journal of Child Psychology and Psychiatry 57: 152-160 (2016).

Yin Y, et al. Impact of cytosine methylation on DNA binding specificities of human transcription factors. Science 356: eaaj2239 (2017).

Yokoi K, et al. Analysis of DNA methylation status in bodily fluids for early detection of cancer. International Journal of Molecular Sciences 18: 735 (2017).

What is claimed is:

1. A method of detecting donor cell death in a subject receiving foreign biological material from a donor, the method comprising
(a) sequencing cell-free DNA in a biospecimen from the subject;
(b) determining cellular origin of the cell-free DNA by identifying methylation patterns in the sequence of the cell-free DNA and comparing the methylation patterns in the sequence of the cell-free DNA to known methylation patterns associated with different cell types; and
(c) determining source origin of the cell-free DNA by genotyping the cell-free DNA and identifying whether the cell-free DNA originates from the foreign biological material or from the subject;
wherein cell death is detected when the cell-free DNA has both a cellular origin of the type of foreign biological material that was received from the donor, and a source origin of the donor.

2. A method of monitoring a subject's response to receiving foreign biological material from a donor, the method comprising detecting cell death in the subject at one or more time points, or at two or more time points, after receiving the foreign biological material, wherein detection of cell death comprises:
(a) sequencing cell-free DNA in a biospecimen from the subject;
(b) determining cellular origin of the cell-free DNA by identifying methylation patterns in the sequence of the cell-free DNA and comparing the methylation patterns in the sequence of the cell-free DNA to known methylation patterns associated with different cell types; and
(c) determining source origin of the cell-free DNA by genotyping the cell-free DNA and identifying whether the cell-free DNA originates from the foreign biological material or from the subject;
wherein cell death is detected when the cell-free DNA has both a cellular origin of the type of foreign biological material that was received from the donor, and a source origin of the donor.

3. A method of treating donor cell death in a subject receiving foreign biological material from a donor, the method comprising administering a treatment for donor cell death when donor cell death in the subject is detected,
wherein donor cell death is detected by a method comprising:
(a) sequencing cell-free DNA in a biospecimen from the subject;
(b) determining cellular origin of the cell-free DNA by identifying methylation patterns in the sequence of the cell-free DNA and comparing the methylation patterns in the sequence of the cell-free DNA to known methylation patterns associated with different cell types; and
(c) determining source origin of the cell-free DNA by genotyping the cell-free DNA and identifying whether the cell-free DNA originates from the foreign biological material or from the subject;
wherein donor cell death is detected when the cell-free DNA has both a cellular origin of the type of foreign biological material that was received from the donor, and a source origin of the donor.

4. A method of treating donor cell death in a subject receiving foreign biological material from a donor, the method comprising administering a treatment for donor cell death when the quantity of donor cell death is increased between two or more time points after the subject receives the foreign biological material,
wherein donor cell death is quantified by a method comprising:
(i) detecting donor cell death in the subject, wherein detection of donor cell death comprises:
(a) sequencing cell-free DNA in a biospecimen from the subject;
(b) determining cellular origin of the cell-free DNA by identifying methylation patterns in the sequence of the cell-free DNA and comparing the methylation patterns in the sequence of the cell-free DNA to known methylation patterns associated with different cell types; and
(c) determining source origin of the cell-free DNA by genotyping the cell-free DNA and identifying whether the cell-free DNA originates from the foreign biological material or from the subject;
wherein donor cell death is detected when the cell-free DNA has a cellular origin of the type of foreign biological material that was received from the donor, and has a source origin of the donor; and
(ii) quantifying the cell-free DNA that has both a cellular origin of the type of foreign biological material that was received from the donor, and a source origin of the donor.

5. The method of any one of claims 1-4, wherein the biospecimen comprises a biological fluid.

6. The method of claim 5, wherein the biological fluid is selected from blood, serum, plasma, cerebrospinal fluid, saliva, urine, and sputum.

7. The method of claim 6, wherein the biological fluid comprises blood, serum, or plasma.

8. The method of any one of claims 1-7, wherein the foreign biological material comprises liver tissue, cardiac tissue, vascular tissue, pancreatic tissue, splenic tissue, esophageal tissue, gastric tissue, intestinal tissue, colon tissue, lung tissue, tracheal tissue, skin tissue, subcutaneous tissue, hair tissue, kidney tissue, connective tissue, muscular tissue, skeletal tissue, cartilage tissue, prostate tissue, bladder tissue, gonadal tissue, uterine tissue, penile tissue, neural tissue, corneal tissue, ophthalmologic tissue, bone marrow tissue, and a population of blood-derived cells.

9. The method of claim 8, wherein the foreign biological material comprises liver tissue.

10. The method of any one of claims 1-9, wherein the methylation pattern comprises a segment of nucleotide sequence containing at least 3 CpG dinucleotides.

11. The method of any one of claims 1-10, wherein the cell types are selected from mature B-cell, naïve B-cell, biliary epithelial cell, breast basal cell, breast luminal cell, bulk endothelial cell, bulk epithelial cell, bulk immune cell, cardiomyocyte, cardiopulmonary endothelial cell, colon epithelial cell, dermal epithelial cell, granulocyte, hepatocyte, keratinocyte, kidney epithelial cell, liver endothelial cell, liver stromal cell, liver resident immune cell, lung epithelial cell, megakaryocyte, monocyte, macrophage, neuron, natural killer cell, pancreatic cell, prostate epithelial cell, skeletal muscular cell, and mature T-cell.

12. The method of any one of claims 1-10, wherein the known methylation patterns are set forth in Table 2.

13. The method of any one of claims 1-12, wherein genotyping the cell-free DNA comprises obtaining a polymorphic marker profile of the cell-free DNA and comparing it to a polymorphic marker profile obtained from the subject or the donor.

14. The method of claim 13, wherein the polymorphic marker profile comprises polymorphic markers selected from single nucleotide polymorphisms, restriction fragment length polymorphisms, variable number of tandem repeats, short tandem repeats, hypervariable regions, minisatellites, dinucleotide repeats, trinucleotide repeats, tetranucleotide repeats, and simple sequence repeats.

15. The method of claim 14, wherein the polymorphic marker profile comprises polymorphic markers selected from single nucleotide polymorphisms

16. The method of any one of claims 1-15, wherein determining cellular origin of the cell-free DNA comprises identifying methylation patterns in one or more portions of the sequence of the cell-free DNA.

17. The method of claim 16, wherein the polymorphic profile is obtained for the same one or more portions of the sequence of the cell-free DNA of which methylation patterns were identified.

18. The method of claim 17, wherein cell death is detected when the one or more portions of cell-free DNA has both a cellular origin of the type of foreign biological material that was received from the donor, and a source origin of the donor.

19. The method of any one of claims 3-18, wherein the treatment comprises an immunosuppressive agent, an anti-inflammatory agent, an antibacterial therapy, an antiviral therapy, or a therapy targeted to a pathway that controls cell death.

20. The method of any one of claims 4-19, wherein the cell-free DNA is quantified using chromatography, electrophoresis, comparative genomic hybridization, microarrays, or bead arrays.

21. The method of any one of claims 4-20, wherein the increase in quantity of donor cell death between the two or more time points is at least 2-fold.

22. The method of any one of claims 2 and 4-21, wherein the two or more time points are two or more days between Day 0 and Day 60 or at later time points with symptoms of tissue dysfunction after the subject receives the foreign biological material.
